(19) United States
(12) Patent Application Publication
(10) Pub. No.: US 2003/0113830 A1
(43) Pub. Date: Jun. 19, 2003

(54) Novel antioxidant, nucleic acid constructs encoding same, pharmaceutical compositions containing same and use of same for reducing oxidative-stress

(75) Inventor: Andrew P. Levy, Kiryat Shmuel (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Rappaport Family Institute for Research in the Medical Sciences

(21) Appl. No.: 09/903,463

(22) Filed: Jul. 11, 2001

(51) Int. Cl. C12Q 1/26; A61K 38/41; C07K 14/795

(52) U.S. Cl. 435/25; 514/6; 530/385

(57) ABSTRACT

Novel haptoglobin derived antioxidants, nucleic acid constructs encoding same, pharmaceutical compositions containing the novel antioxidant or the nucleic acid constructs, and methods of relieving oxidative stress by administration of the antioxidants, the nucleic acid constructs encoding same or the pharmaceutical composition containing same to a subject in need thereof are disclosed.

### FIELD AND BACKGROUND OF THE INVENTION

The present invention relates to a novel haptoglobin derived antioxidants, nucleic acid constructs encoding same, pharmaceutical compositions containing the novel antioxidant or the nucleic acid constructs, and further to methods of relieving oxidative stress by administration of the antioxidants, the nucleic acid constructs encoding same or the pharmaceutical composition containing same to a subject in need thereof. The present invention further relates to a method of evaluating a potential of a haptoglobin derived polypeptide in reducing oxidation induced by oxygenized hemoglobin

Haptoglobin is a serum protein which functions as an antioxidant by virtue of its ability to bind to hemoglobin [1] and thereby preventing the oxidative tissue damage which may be mediated by free hemoglobin [2]. The importance of this protective mechanism has been demonstrated in haptoglobin knockout mice which develop a marked increase in oxidative tissue damage in response to hemolysis [3]. In man there exists two alleles (denoted 1 and 2) for the haptoglobin gene [1, 2, 4]. The biophysical and biochemical properties of the haptoglobin polymeric molecules resulting from the three possible combinations (haptoglobin 1-1, 2-1 or 2-2) of these two alleles are dramatically different.²

It was recently found in multiple independent studies [5-8] (and Levy A P, Hochberg I, Jablonski K, et al. manuscript submitted for publication) of over 1000 individuals from Israel, Belgium and the United States that the haptoglobin phenotype is a predictor of the risk of developing both microvascular and macrovascular complications of diabetes. Specifically, diabetic individuals with the haptoglobin 1-1 phenotype were shown to be remarkably resistant to the development of diabetic retinopathy, diabetic nephropathy, and cardiovascular disease [5-8]. Moreover, it was found that there was a graded effect evident with regard to risk and the number of haptoglobin 2 alleles [7,8]. For example, in a prospective study of incident cardiovascular disease, it was found that individuals homozygous for the haptoglobin 2 allele had a 5 fold increase in the risk of cardiovascular disease compared to individuals homozygous for the haptoglobin 1 allele, while heterozygotes were found to have an intermediate risk (Levy A P, Hochberg I, Jablonski K, et al. manuscript submitted for publication). Further details in this respect are disclosed in U.S. Pat. No. 6,251,608; U.S. patent application Ser. Nos. 09/688,121; and 09/815,016 and in PCT Application Nos. IL00/00359; IL01/00368; and IL01/00369, which are incorporated by reference as if fully set forth herein.

An increase in oxidative stress has been proposed to play a crucial role in the development of diabetic vascular complications [9-10]. Accordingly, differences in the genetically endowed antioxidant status may confer increased or decreased susceptibility to the development of these diabetic vascular complications.

A key site of action of haptoglobin in neutralizing the oxidative capacity of hemoglobin is the extravascular space, particularly after endothelial injury. Haptoglobin 1-1 and 2-2 clearly differ in their ability to sieve into the extravascular compartment across the endothelial cell barrier [2]. Since this difference in sieving is possibly a reflection of the profound differences in the size of haptoglobin 1-1 dimers and haptoglobin 2-2 cyclic polymers it was sought to identify a minimal haptoglobin peptide with preserves antioxidant function and which would have an improved ability to penetrate into the extravascular space, assuming that such a minimal haptoglobin peptide, if isolateable, would serve to augment the anti-oxidative capabilities in vivo in subjects in need thereof.

### SUMMARY OF THE INVENTION

Haptoglobin serves as an antioxidant by virtue of its ability to prevent hemoglobin driven oxidative tissue damage. It was recently demonstrated that an allelic polymorphism in the haptoglobin gene is predictive of the risk of developing numerous microvascular and macrovascular diabetic complications. Since these complications are attributed in large part to an increase in oxidative stress, it was sought to determine whether the different protein products of the two haptoglobin alleles differed in the antioxidant protection that they provided. A statistically significant difference was found in the antioxidant capacity of purified haptoglobin protein produced from the two different alleles, consistent with the hypothesis that differences in the genetically determined antioxidant status may explain differential susceptibility to diabetic vascular complications. These differences may be amplified in the vessel wall due to differences in the sieving capacity of the haptoglobin types. It was therefore further sought to identify the minimal haptoglobin sequences necessary to inhibit oxidation by hemoglobin. Several independent haptoglobin peptides that function in this fashion as efficiently as native haptoglobin were identified.

According to one aspect of the present invention there is provided a method of evaluating a potential of a haptoglobin derived polypeptide in reducing oxidation induced by oxygenized hemoglobin, the method comprising reacting hemoglobin with an oxidizable substrate in a presence and an absence, and/or in a presence of varying concentrations of the haptoglobin derived polypeptide; and determining an effect of the presence and the absence, and/or the presence of the varying concentrations of the haptoglobin derived polypeptide on oxidation of the oxidizable substrate, thereby evaluating the potential of the haptoglobin derived polypeptide in reducing the oxidation induced by the hemoglobin.

According to further features in preferred embodiments of the invention described below, the oxidizable substrate comprises a fatty acid.

According to still further features in the described preferred embodiments the oxidizable substrate comprises a fatty acid.

According to still further features in the described preferred embodiments the oxidizable substrate comprises an unsaturated (poly or monounsaturated) fatty acid, preferably a long unsaturated fatty acid, having a carbon backbone of at least 10, preferably at least 12, more preferably between 12 and 24 carbon atoms in its backbone, such as, for example, linolenic acid.

According to still further features in the described preferred embodiments the oxidizable substrate comprises low density lipoprotein (LDL).

According to still further features in the described preferred embodiments the oxidizable substrate comprises very low density lipoprotein (VLDL).

According to still further features in the described preferred embodiments the oxidizable substrate comprises chylomicrons.

According to still further features in the described preferred embodiments determining the effect is by monitoring at least one oxidation product of the oxidizeable substrate.

According to still further features in the described preferred embodiments the oxidation product comprises conjugated dienes.

According to still further features in the described preferred embodiments monitoring the at least one oxidation product of the oxidizeable substrate is effected spectrally.

According to another aspect of the present invention there is provided an antioxidant compound comprising a polypeptide having an amino acid sequence derived from an alpha subunit of a haptoglobin protein sequence, the polypeptide being capable of reducing oxidation induced by oxygenized hemoglobin, the antioxidant compound being free of amino acid sequences derived from a beta subunit of a haptoglobin protein. Preferably, the polypeptide has an amino acid sequence derived from a portion of an alpha subunit of a haptoglobin protein sequence, the portion being capable of reducing oxidation induced by oxygenized hemoglobin.

According to yet another aspect of the present invention there is provided an antioxidant compound comprising a polypeptide having an amino acid sequence derived from a portion of an alpha subunit of a haptoglobin protein sequence, the polypeptide being capable of reducing oxidation induced by oxygenized hemoglobin, the polypeptide being free of remaining portions of the alpha subunit of the haptoglobin protein sequence. Preferably, the polypeptide has an amino acid sequence derived from a consecutive portion of the alpha subunit of a haptoglobin protein sequence, the portion being capable of reducing oxidation induced by oxygenized hemoglobin.

According to further features in preferred embodiments of the invention described below, the polypeptide is as set forth in SEQ ID NOs:19 or 20.

According to still another aspect of the present invention there is provided an antioxidant compound comprising a polypeptide having an amino acid sequence derived from a beta subunit of a haptoglobin protein sequence, the polypeptide being capable of reducing oxidation induced by oxygenized hemoglobin, the antioxidant compound being free of amino acid sequences derived from an alpha subunit of a haptoglobin protein. Preferably, the polypeptide has an amino acid sequence derived from a portion of the beta subunit of a haptoglobin protein sequence, the portion being capable of reducing oxidation induced by oxygenized hemoglobin.

According to an additional aspect of the present invention there is provided an antioxidant compound comprising a polypeptide having an amino acid sequence derived from a portion of a beta subunit of a haptoglobin protein sequence, the polypeptide being capable of reducing oxidation induced by oxygenized hemoglobin, the polypeptide being free of remaining portions of the beta subunit of a haptoglobin protein sequence. Preferably, the polypeptide has an amino acid sequence derived from a consecutive portion of a beta subunit of a haptoglobin protein sequence, the portion being capable of reducing oxidation induced by oxygenized hemoglobin.

According to further features in preferred embodiments of the invention described below, the polypeptide is as set forth in SEQ ID NOs:15 or 16.

According to yet an additional aspect of the present invention there is provided a nucleic acid construct comprising a first polynucleotide encoding a polypeptide having an amino acid sequence derived from an alpha subunit of a haptoglobin protein sequence, the polypeptide being capable of reducing oxidation induced by oxygenized hemoglobin, the polynucleotide being free of amino acid sequences derived from a beta subunit of a haptoglobin protein; and a second polynucleotide harboring a promoter operably linked to the first polynucleotide.

According to still an additional aspect of the present invention there is provided a nucleic acid construct comprising a first polynucleotide encoding a polypeptide having an amino acid sequence derived from a portion of an alpha subunit of a haptoglobin protein sequence, the polypeptide being capable of reducing oxidation induced by oxygenized hemoglobin, the polypeptide being free of remaining portions of the alpha subunit of the haptoglobin protein sequence; and a second polynucleotide harboring a promoter operably linked to the first polynucleotide.

According to further features in preferred embodiments of the invention described below, the first polynucleotide is as set forth in SEQ ID NOs:13 or 14.

According to a further aspect of the present invention there is provided a nucleic acid construct comprising a first polynucleotide encoding a polypeptide having an amino acid sequence derived from a beta subunit of a haptoglobin protein sequence, said polypeptide being capable of reducing oxidation induced by oxygenized hemoglobin, the antioxidant compound being free of amino acid sequences derived from an alpha subunit of a haptoglobin protein; and a second polynucleotide harboring a promoter operably linked to said first polynucleotide.

According to still a further aspect of the present invention there is provided a nucleic acid construct comprising a first polynucleotide encoding a polypeptide having an amino acid sequence derived from a portion of a beta subunit of a haptoglobin protein sequence, the polypeptide being capable of reducing oxidation induced by oxygenized hemoglobin, the polypeptide being free of remaining portions of the beta subunit of a haptoglobin protein sequence; and a second polynucleotide harboring a promoter operably linked to the first polynucleotide.

According to further features in preferred embodiments of the invention described below, the first polynucleotide is as set forth in SEQ ID NO:9, 10.

The haptoglobin protein sequence is preferably of a mammal, such as human, mouse, rat and dog.

According to another aspect of the present invention there is provided a pharmaceutical composition comprising, as an active ingredient, the antioxidant compound or nucleic acid construct described herein and a pharmaceutically acceptable carrier.

According to further features in preferred embodiments of the invention described below, the pharmaceutical composition is packaged and identified as containing an antioxidant.

According to still further features in the described preferred embodiments the pharmaceutical composition is packaged and identified for use in relieving oxidative stress.

According to still further features in the described preferred embodiments the pharmaceutical composition is packaged and identified for use in a pathology or habit associated with elevated oxidative stress.

According to still further features in the described preferred embodiments the pharmaceutically acceptable carrier comprises a solid support.

According to still further features in the described preferred embodiments the solid support is a stent.

According to still further features in the described preferred embodiments the pharmaceutically acceptable carrier is designed for slow release.

According to yet another aspect of the present invention there is provided a method of reducing oxidative stress in a subject in need, the method comprising administering to the subject an antioxidant compound or a nucleic acid construct as described herein per se or as an active ingredient of a pharmaceutical composition that may further include a pharmaceutically acceptable carrier as described herein. The reason for thus treating the individual may be associated with either a pathology and/or a habit.

The present invention successfully addresses the shortcomings of the presently known configurations by providing novel means with which to combat oxidative stress, which is responsible for many of the aliments of human beings in western society.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 presents a schematic map of the haptoglobin subunits and truncated mutants. Native haptoglobin (hatched) is made as a single polypeptide and then cleaved into an alpha and beta subunit which are joined by disulfide bonds to form a haptoglobin monomer. The two alleles for haptoglobin differ only in their alpha subunit. The RHp constructs were made as described in methods and correspond to the amino acids of the beta or alpha subunit as shown. RHp 1 is the entire beta subunit. RHp2-4 are truncated mutants of the beta subunit. RHp5 is the alpha subunit from the 2 allele and RHp6 is the alpha subunit from the 1 allele.

FIGS. 2a-b demonstrate oxidation of linolenic acid by hemoglobin. FIG. 2a is a plot demonstrating the time-dependent increase in conjugated diene (absorbace at 232 nm, A232) formation when linolenic acid is incubated with hemoglobin in the presence of no haptoglobin, haptoglobin 1-1 (0.6 μM) or haptoglobin 2-2 (0.6 μM) as described in the Examples section that follows. Data shown are the mean+/−SME for nine independent experiments. FIG. 2b is a bar graph comparing the percent inhibition of hemoglobin-induced oxidation of linolenic acid by haptoglobin 1-1 or 2-2. Hemoglobin was used at a concentration of 0.62 μM and haptoglobin was used at a concentration of 0.6 μM. Data for two types of haptoglobin are expressed as the percent inhibition of oxidation that occurred in a reaction performed in parallel in the absence of any haptoglobin at the 60 minute time point. Data shown are the mean+/−SME of nine independent experiments. The difference in the mean % inhibition between haptoglobin 1-1 and haptoglobin 2-2 was statistically significant (p<0.05).

FIGS. 3a-b demonstrate oxidation of LDL by hemoglobin. FIG. 3a is a plot demonstrating the time-dependent increase in TBARS formation when LDL is incubated with hemoglobin (10 μM) in the presence of no haptoglobin, haptoglobin 1-1 (5 μM) or haptoglobin 2-2 (5 μM) as described in the Examples section that follows. Data shown are the mean+/−SME for four independent experiments. FIG. 3b is a bar graph comparing the ability to inhibit the hemoglobin-induced oxidation of LDL by haptoglobin 1-1 or 2-2. Hemoglobin was used at a concentration of 10 μM and haptoglobin was used at a concentration of 5 μM. Data are expressed as a percentage of inhibition of the amount of TBARS obtained in the absence of any haptoglobin over the entire incubation period by integrating the area under the TBARS vs. time curve using MATLAB as described in the Examples section that follows. Data shown are the mean+/−SME of four independent experiments. The difference in the mean % inhibition between haptoglobin 1-1 and haptoglobin 2-2 was statistically significant (p<0.004).

FIGS. 4a-b demonstrate that truncated haptoglobin inhibits the oxidation of linolenic acid by hemoglobin. Recombinant haptoglobin was produced as described in the Examples section that follows. FIG. 4a is a bar graph demonstrating the lack of inhibition of oxidation of linolenic acid by a 40 amino acid construct RHp3 (derived from RHp2). FIG. 4b is a bar graph demonstrating concentration dependent inhibition of oxidation of linolenic acid by a 81 amino acid construct derived from the haptoglobin beta subunit (RHp2).

FIG. 5 shows a schematic map of the different shapes of the haptoglobin polymers as determined by phenotype. These shapes have been confirmed by electron microscopic analysis of haptoglobin purified from patients with haptoglobin 1-1, 2-1 or 2-2 [20]. Critical disulfide linkages necessary for covalent cross linking of haptoglobin monomers (circles) to form polymers are found on exons 3 and 4 (alpha subunit of haptoglobin). The haptoglobin 2 allele has a duplication of exons 3 and 4. Haptoglobin 1 monomer is univalent (note single arrow) and thus can only associate with one other haptoglobin molecule to create dimers. Haptoglobin 2 monomer is bivalent (note two arrows) and can associate with two different haptoglobin monomers. Consequently the haptoglobin in individuals homozygous for the 2 allele will be cyclic polymers.

### DESCRIPTION OF THE PREFERRED EMBODIMENTS

The present invention is of novel haptoglobin derived antioxidants, nucleic acid constructs encoding same, pharmaceutical compositions containing the novel antioxidant or the nucleic acid constructs, and methods of relieving oxidative stress by administration of the antioxidants, the nucleic acid constructs encoding same or the pharmaceutical composition containing same to a subject in need thereof. The present invention is further of a method of evaluating a potential of a haptoglobin derived polypeptide in reducing oxidation induced by oxygenized hemoglobin. Specifically, the present invention can be used to augment the antioxidation capacity of serum of individuals having haptoglobin type 2, which has poorer antioxidation capabilities as compared to haptoglobin type 1. Most specifically, the present invention can be used to augment the antioxidation capacity of serum of individuals having haptoglobin type 2, which has poorer antioxidation capabilities as compared to haptoglobin type 1 and which are diabetic and hence are exposed to elevated oxidative stress due to hyperglycemia.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Previous epidemiological studies have demonstrated that the haptoglobin type is fundamentally important in the development of diabetic vascular disease.

While reducing the present invention to practice it was demonstrated that there are functional differences in the antioxidant capacity of the different haptoglobin proteins towards hemoglobin suggesting that individuals with haptoglobin 1-1 protein may have superior antioxidant protection compared to individuals with haptoglobin 2-2 protein. These data are consistent with earlier reports showing that the consumption of vitamin C in the plasma in vitro of individuals with haptoglobin 2-2 was more rapid than in the plasma of individuals with haptoglobin 1-1, and that vitamin C levels are significantly lower in individuals with haptoglobin 2-2 [19].

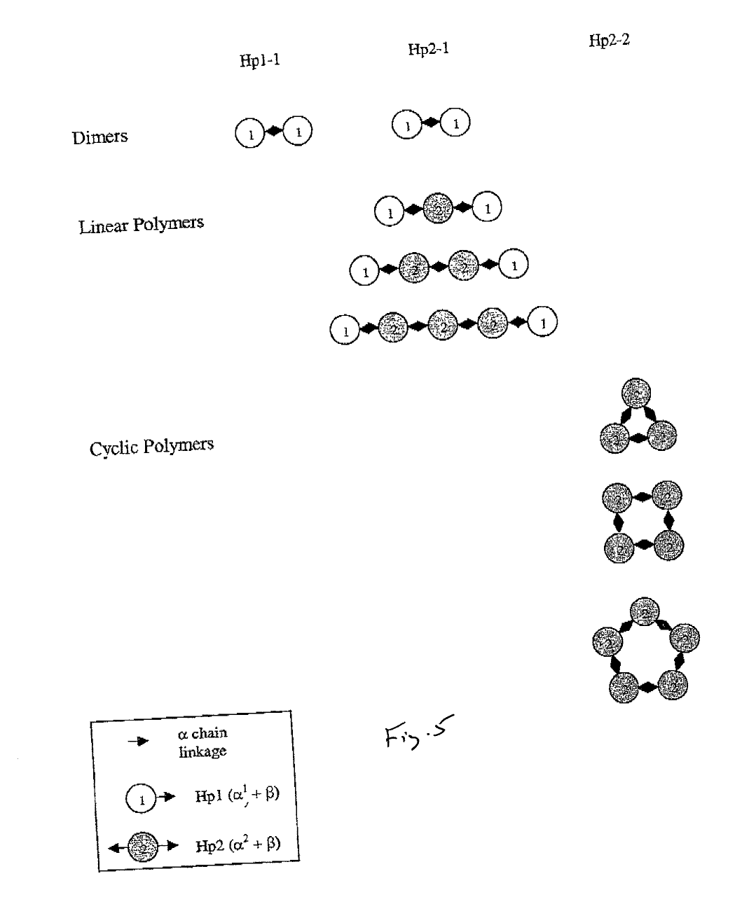

The statistically significant but relatively modest differences described herein in anti-oxidant capacity between the different haptoglobin types may be dramatically amplified in vivo due to differences in the ability of the different haptoglobin types to gain access to the vessel wall. A schematic drawing of the different haptoglobin polymers [1, 20] in individuals with haptoglobin 1-1 or 2-2 as shown in FIG. 5, demonstrates the large differences in size between the different haptoglobin types that are likely to account for these differences in sieving capacity. At sites of blood vessel injury (e.g., following coronary angioplasty) there is a sudden release of free hemoglobin into the blood vessel wall. Haptoglobin is not normally found in appreciable concentrations in the normal vessel wall. Therefore, the ability of haptoglobin to sieve into the vessel wall in order to neutralize hemoglobin is likely to be of great importance. In the diabetic individual, already burdened with increased oxidative stress due to hyperglycemia [9, 10], differences in genetically determined endogenous antioxidant protection may have exaggerated importance.

Further while reducing the present invention to practice, two peptides derived from haptoglobin were identified that can independently bind to hemoglobin and prevent it from oxidizing substrate as effectively as the full-length haptoglobin molecule. Such a mini-haptoglobin is expected to have improved access to the extravascular space and thus serve as superior antioxidants over endogenous haptoglobin.

Haptoglobin-hemoglobin complexes have recently been demonstrated to be specifically taken up by a receptor-mediated mechanism (CD163) by macrophages in a phenotype-dependent fashion [21]. The CD163 receptor was shown to have a 10-fold higher affinity for haptoglobin 2-2 as compared to haptoglobin 1-1 [21]. Differences in the receptor-mediated endocytosis of the hemoglobin-haptoglobin complex would be expected to lead to an increase in intracellular iron-induced oxidative stress in individuals with haptoglobin 2-2 versus haptoglobin 1-1. Indeed, Langlois et al. have demonstrated that serum iron and monocyte L-ferritin concentrations in haptoglobin 2-2 individuals are dramatically higher than in individuals with haptoglobin 1-1 [22]. A differential effect on intracellular oxidative stress between haptoglobin 2-2 and 1-1 may be amplified as the haptoglobin 2-2 hemoglobin complex is more efficiently taken up by the cells. This potential source of oxidative stress (mediated by CD163 and the haptoglobin-hemoglobin complex) may only become a real source of oxidative stress in situations in which the individual is already experiencing oxidative stress for an additional reason such as a pathology, e.g., diabetes, and/or a habit, e.g., smoking, nutrition, extensive sun tanning, etc.

According to one aspect of the present invention there is provided a method of evaluating a potential of a haptoglobin derived polypeptide in reducing oxidation induced by oxygenized hemoglobin. The method according to this aspect of the invention is effected by reacting hemoglobin with an oxidizable substrate in the presence and absence, and/or in the presence of varying concentrations of the haptoglobin derived polypeptide, and determining the effect of the presence and the absence, and/or the presence of the varying concentrations of the haptoglobin derived polypeptide on oxidation of the oxidizable substrate, thereby evaluating the potential of the haptoglobin derived polypeptide in reducing the oxidation induced by the hemoglobin.

As used herein, the terms “polypeptide” and “peptide” are used interchangeably and include native peptides (either degradation products, synthetically synthesized peptides or recombinant peptides) and peptidomimetics (typically, synthetically synthesized peptides), such as peptoids and semipeptoids which are peptide analogs, which may have, for example, modifications rendering the peptides more stable while in a body, or less immunogenic. Such modifications include, but are not limited to, cyclization, N-terminus modification, C-terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modification and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further detail in this respect are provided hereinunder.

Thus, a peptide according to the present invention can be a cyclic peptide. Cyclization can be obtained, for example, through amide bond formation, e.g., by incorporating Glu, Asp, Lys, Om, di-amino butyric (Dab) acid, di-aminopropionic (Dap) acid at various positions in the subunit (—CO—NH or —NH—CO bonds). Backbone to backbone cyclization can also be obtained through incorporation of modified amino acids of the formulas H—N((CH₂)ₙ—COOH)—C(R)H—COOH or H—N((CH₂)ₙ—COOH)—C(R)H—NH₂, wherein n=1-4, and further wherein R is any natural or non-natural side subunit of an amino acid.

Cyclization via formation of S—S bonds through incorporation of two Cys residues is also possible. Additional side-subunit to side subunit cyclization can be obtained via formation of an interaction bond of the formula —(—CH₂—)ₙ—S—CH₂—C—, wherein n=1 or 2, which is possible, for example, through incorporation of Cys or homoCys and reaction of its free SH group with, e.g., bromoacetylated Lys, Om, Dab or Dap.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), o-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side subunit, naturally presented on the carbon atom.

These modifications can occur at any of the bonds along the peptide subunit and even at several (2-3) at the same time.

Natural aromatic amino acids, Trp, Tyr and Phe, may be substituted for synthetic non-natural acid such as TIC, naphthyl (Nol), ring-methylated derivatives of Phe, halogenated derivatives of Phe or o-methyl-Tyr.

Accordingly, as used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids which are linked via a peptide bond or a peptide bond analog to at least one addition amino acid as this term is defined herein.

An amino acid residue is understood to be an amino acid as this term is defined herein when serving as a building block or unit in a peptide, as this term is defined herein.

Tables 1-2 below list all the naturally occurring amino acids (Table 1) and non-conventional or modified amino acids (Table 2).

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)gIycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycme | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

As used herein, the phrase “haptoglobin derived polypeptide” which is also referred to herein as a “polypeptide having an amino acid sequence derived from an alpha (or beta) subunit of a haptoglobin protein sequence” includes peptides as this term is defined hereinabove which are identical or similar in their amino acid sequence to at least a portion of a haptoglobin alpha (or beta) subunit. The level of similarity between the haptoglobin derived polypeptide and haptoglobin will be discussed hereinbelow.

The oxidizable substrate that is used in the method of evaluating a potential of a haptoglobin derived polypeptide in reducing oxidation induced by oxygenized hemoglobin according to the present invention is preferably an unsaturated (poly or monounsaturated) fatty acid, preferably a long unsaturated fatty acid, having a carbon backbone of at least 10, preferably at least 12, more preferably between 12 and 24 carbon atoms in its backbone, such as, for example, linolenic acid. Low density lipoprotein (LDL), very low density lipoprotein (VLDL) or chylomicrons, which comprise unsaturated fatty acid can also serve as oxidizable substrates in accordance with the teachings of the present invention. These are the preferred substrates because these substrates are in many cases the target for hemoglobin induced oxidation in the body. Also, oxidation products of these targets (e.g., conjugated dienes) are monitorable directly or indirectly via spectrometric methods which are efficient and highly quantitative over a wide dynamic range.

The method of evaluating a potential of a haptoglobin derived polypeptide in reducing oxidation induced by oxygenized hemoglobin according to the present invention can be readily used to systematically test any haptoglobin derived polypeptide, as this term is defined herein, for its potential in reducing oxidation induced by oxygenized hemoglobin. The haptoglobin derived polypeptide that can be tested using this method is identical similar or similar to a haptoglobin protein sequence of a mammal, such as human, mouse, rat and dog. The human haptoglobin alpha and beta subunit amino acid sequences are listed in SEQ ID NOs:19 and 15, respectively. Haptoglobin alpha and beta subunits amino acid sequences are obtainable from, e.g., GB (GenBank) and EMBL (European Molecular Biology Laboratory) and are listed, identified by an accession No., along with the accession No. of the respective haptoglobin gene (if available) in Table 3 below:

TABLE 3

| | Hp alpha subunit | Hp beta subunit | Hp gene |
|---|---|---|---|
| Species | aa sequence | aa sequence | sequence |
| Mouse | GB | GB | GB |
| | AAA37778 | AAA37778 | NM_017370 |
| Rat | GB | GB | GB |
| | AAA41348.1 | AAA41348.1 | NM 012582.1 |
| Human (1 allele) | EMBL | EMBL | GB |
| | CAA25267.1 | CAA25267.1 | X 00637.1 |
| Human (2 allele) | EMBL | EMBL | GB |
| | CAA25248.1 | CAA25248.1 | AH 003344.1 |
| dog | GB | GB | — |
| | AAB23858.1 | AAB23859.1 | |

GB = Genbank;
EMBL = European Molecular Biology Laboratory

A haptoglobin derived polypeptide according to the present invention can be identical or similar to the amino acid sequence of a haptoglobin alpha or beta subunit. The level of similarity may be at least 70%, at least 75%, at least 80%, at least 85%, at least 90%, at least 95%, preferably between 95% and 100%. The level of similarity can be determined using any sequence homology software such as, for example, the GCG software.

A haptoglobin derived polypeptide according to the present invention can have a length of, for example, 10 to 245 amino acids, preferably, at least 20 amino acids, at least 25 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 95 amino acids, at least 100 amino acids, at least 105 amino acids, at least 110 amino acids, at least 115 amino acids, at least 120 amino acids, at least 125 amino acids, at least 130 amino acids, vat least 135 amino acids, at least 140 amino acids, at least 145 amino acids, at least 150 amino acids, at least 155 amino acids, at least 160 amino acids, at least 165 amino acids, at least 170 amino acids, at least 175 amino acids, at least 180 amino acids, at least 190 amino acids, at least 200 amino acids, at least 20 amino acids, at least 210 amino acids, at least 220 amino acids, at least 230 amino acids, at least 240 amino acids, or at least 245, it can be derived from either the alpha or the beta subunit of a haptoglobin, it can be a purified proteolytic product of a natural haptoglobin, produced by recombinant techniques, or be synthesized using solid phase techniques. Using each of these techniques an ordinary technician can test thousands of haptoglobin derived polypeptide in a short time period. This can be used to obtain a haptoglobin derived polypeptide of superior qualities in reducing hemoglobin induced oxidation, which can thereafter be further tested in vivo for pharmacokinetic and therapeutic properties. Once a haptoglobin derived polypeptide of superior qualities in reducing hemoglobin induced oxidation is identified and its pharmacokinetic and therapeutic properties are evaluated it be modified in a process known as drug optimization, so as to acquire even better antioxidation properties.

Hence, according to another aspect of the present invention there is provided an antioxidant compound comprising a polypeptide having an amino acid sequence derived from an alpha subunit of a haptoglobin protein sequence. The polypeptide is capable of reducing oxidation induced by oxygenized hemoglobin. The antioxidant compound according to this aspect of the present invention is free of amino acid sequences derived from a beta subunit of a haptoglobin protein. As such, it is distinct from natural haptoglobin, or a proteolysate of haptoglobin, as described, for example, in reference [18]. Preferably, the polypeptide has an amino acid sequence derived from a portion of an alpha subunit of a haptoglobin protein sequence, that portion is capable of reducing oxidation induced by oxygenized hemoglobin, as is determined, for example, using the method of evaluating a potential of a haptoglobin derived polypeptide in reducing oxidation induced by oxygenized hemoglobin of the present invention, which is described herein above and is further exemplified in the Examples section that follows.

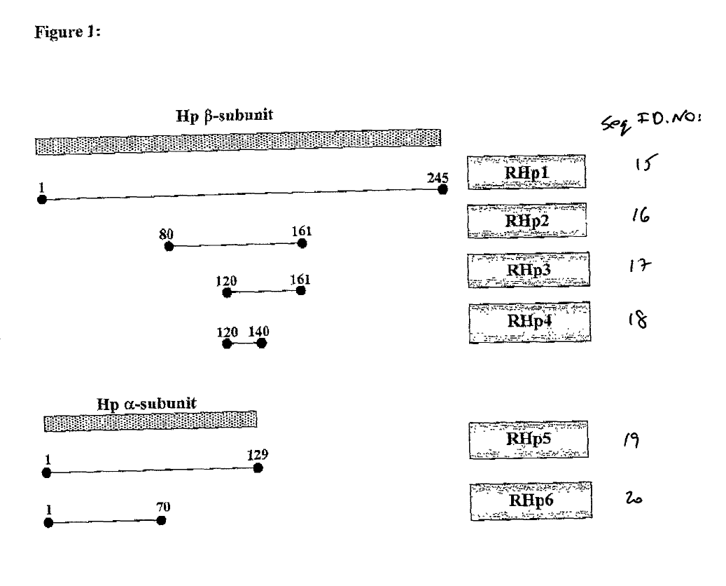

As used herein the term portion refers to a part of a whole, i.e., a segment. Thus, for example, a portion of the human haptoglobin alpha subunit may include up to 128 amino acids, whereas a portion of the human haptoglobin beta subunit may include up to 244 amino acids (see FIG. 1).

According to yet another aspect of the present invention there is provided an antioxidant compound comprising a polypeptide having an amino acid sequence derived from a portion of an alpha subunit of a haptoglobin protein sequence. The polypeptide according to this aspect of the present invention is capable of reducing oxidation induced by oxygenized hemoglobin. The polypeptide according to this aspect of the present invention is free of remaining portions of the alpha subunit of the haptoglobin protein sequence. Preferably, the polypeptide according to this aspect of the present invention has an amino acid sequence derived from a consecutive portion of the alpha subunit of a haptoglobin protein sequence, the portion being capable of reducing oxidation induced by oxygenized hemoglobin. An example include the polypeptide set forth in SEQ ID NO:19, which includes a portion of amino acids 1-70 of the human haptoglobin alpha subunit, and is free of remaining potions of the human haptoglobin alpha subunit, i.e., amino acids 71-29 of SEQ ID NO:20.

According to still another aspect of the present invention there is provided an antioxidant compound comprising a polypeptide having an amino acid sequence derived from a beta subunit of a haptoglobin protein sequence. The polypeptide according to this aspect of the present invention is capable of reducing oxidation induced by oxygenized hemoglobin. The antioxidant compound according to this aspect of the present invention is free of amino acid sequences derived from an alpha subunit of a haptoglobin protein. Preferably, the polypeptide has an amino acid sequence derived from a portion of the beta subunit of a haptoglobin protein sequence, this portion is capable of reducing oxidation induced by oxygenized hemoglobin.

According to an additional aspect of the present invention there is provided an antioxidant compound comprising a polypeptide having an amino acid sequence derived from a portion of a beta subunit of a haptoglobin protein sequence. The polypeptide according to this aspect of the present invention is capable of reducing oxidation induced by oxygenized hemoglobin. The polypeptide according to this aspect of the present invention is free of remaining portions of the beta subunit of a haptoglobin protein sequence. Preferably, the polypeptide has an amino acid sequence derived from a consecutive portion of a beta subunit of a haptoglobin protein sequence, this portion is capable of reducing oxidation induced by oxygenized hemoglobin. Examples for a polypeptide according to this aspect of the present invention include those polypeptides represented by SEQ ID NOs:15 and 16, wherein SEQ ID NO:15 has the entire 245 amino acids of the human haptoglobin beta subunit, whereas SEQ ID NO: 16 has only amino acids 80-161 (total of 81 amino acids), yet is free of the remaining portions of the human haptoglobin beta subunit, i.e., amino acids 1-79 and 162-245.

As stated hereinabove, the polypeptides of the present invention can be synthesized using solid phase techniques, prepared from natural sources of haptoglobin via protein degradation techniques, such as proteolysis, or produced recombinantly. Recombinantly produced proteins require the construction of nucleic acid constructs adapted for expression in an expression system, such as a bacteria, yeast or a higher cell expression system.

Hence, according to yet an additional aspect of the present invention there is provided a nucleic acid construct comprising a first polynucleotide encoding a polypeptide having an amino acid sequence derived from an alpha subunit of a haptoglobin protein sequence, the polypeptide being capable of reducing oxidation induced by oxygenized hemoglobin, the polynucleotide being free of amino acid sequences derived from a beta subunit of a haptoglobin protein; and a second polynucleotide harboring a promoter operably linked to the first polynucleotide.

According to still an additional aspect of the present invention there is provided a nucleic acid construct comprising a first polynucleotide encoding a polypeptide having an amino acid sequence derived from a portion of an alpha subunit of a haptoglobin protein sequence, the polypeptide being capable of reducing oxidation induced by oxygenized hemoglobin, the polypeptide being free of remaining portions of the alpha subunit of the haptoglobin protein sequence; and a second polynucleotide harboring a promoter operably linked to the first polynucleotide.

SEQ ID NOs:13 and 14 which encode the polypeptides set forth in SEQ ID NOs:19 and 20 provide examples.

According to a further aspect of the present invention there is provided a nucleic acid construct comprising a first polynucleotide encoding a polypeptide having an amino acid sequence derived from a beta subunit of a haptoglobin protein sequence, said polypeptide being capable of reducing oxidation induced by oxygenized hemoglobin, the antioxidant compound being free of amino acid sequences derived from an alpha subunit of a haptoglobin protein; and a second polynucleotide harboring a promoter operably linked to said first polynucleotide.

According to still a further aspect of the present invention there is provided a nucleic acid construct comprising a first polynucleotide encoding a polypeptide having an amino acid sequence derived from a portion of a beta subunit of a haptoglobin protein sequence, the polypeptide being capable of reducing oxidation induced by oxygenized hemoglobin, the polypeptide being free of remaining portions of the beta subunit of a haptoglobin protein sequence; and a second polynucleotide harboring a promoter operably linked to the first polynucleotide.

SEQ ID NOs:9 and 10 which encode the polypeptides set forth in SEQ ID NOs:15 and 16 provide examples.

The first polynucleotide of the present invention can be a portion of any coding sequence of a haptoglobin gene, exemplary list thereof is disclosed in Table 1 above. The length of the first polynucleotide according to the present invention can vary, depending on the length of the polypeptide it encodes.

While the first polynucleotide described herein is an essential element of the invention, it is modular and can be used in different contexts. The promoter of choice that is used in conjunction with this invention is of secondary importance, and will comprise any suitable promoter sequence. It will be appreciated by one skilled in the art, however, that it is necessary to make sure that the transcription start site(s) will be located upstream of an open reading frame. In a preferred embodiment of the present invention, the promoter that is selected comprises an element that is active in the particular host cells of interest. These elements may be selected from transcriptional regulators that activate the transcription of genes essential for the survival of these cells in conditions of stress or starvation, including the heat shock proteins.

A construct according to the present invention preferably further includes an appropriate selectable marker. In a more preferred embodiment according to the present invention the construct further includes an origin of replication. In another most preferred embodiment according to the present invention the construct is a shuttle vector, which can propagate both in E. coli (wherein the construct comprises an appropriate selectable marker and origin of replication) and be compatible for propagation in cells, or integration in the genome, of an organism of choice. The construct according to this aspect of the present invention can be provided, for example, as a plasmid, a bacmid, a phagemid, a cosmid, a phage, a virus or an artificial chromosome.

The constructs of the present invention can also be used to deliver gene therapy. Gene therapy as used herein refers to the transfer of genetic material (e.g., DNA or RNA) of interest into a host to treat or prevent a genetic or acquired disease or condition or phenotype. The genetic material of interest encodes a product (e.g., a protein, polypeptide, peptide, functional RNA, antisense) whose production in vivo is desired. For example, the genetic material of interest can encode a hormone, receptor, enzyme, polypeptide or peptide of therapeutic value. For review see, in general, the text “Gene Therapy” (Advanced in Pharmacology 40, Academic Press, 1997).

Two basic approaches to gene therapy have evolved: (i) ex vivo gene therapy and (ii) in vivo gene therapy. In ex vivo gene therapy cells are removed from a patient, and while being cultured are treated in vitro. Generally, a functional gene is introduced into the cell via an appropriate gene delivery vehicle/method (transfection, transformation, homologous recombination, etc.) and an expression system as needed and then the modified cells are expanded in culture and returned to the host/patient. These genetically reimplanted cells have been shown to express the transfected genetic material in vivo.

In in vivo gene therapy, target cells are not removed from the subject rather the genetic material to be transferred is introduced into the cells of the recipient organism in situ, that is within the recipient. In an alternative embodiment, if the host gene is defective, the gene is repaired in situ (Culver, 1998. (Abstract) Antisense DNA & RNA based therapeutics, February 1998, Coronado, Calif.). These genetically altered cells have been shown to express the transfected genetic material in situ.

The gene expression vehicle is capable of delivery/transfer of nucleic acid into a host cell. The expression vehicle may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression vehicle. Therefore, as used herein the expression vehicle may, as needed, not include the 5′UTR and/or 3′UTR of the actual gene to be transferred and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any nontranslated DNA sequence which works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A Laboratory Manual, Cold Springs Harbor Laboratory, New York 1989, 1992), in Ausubel et al., Current Protocols in Molecular Biology, John Wiley and Sons, Baltimore, Md. 1989), Chang et al., Somatic Gene Therapy, CRC Press, Ann Arbor, Mich. 1995), Vega et al., Gene Targeting, CRC Press, Ann Arbor Mich. (995), Vectors: A Survey of Molecular Cloning Vectors and Their Uses, Butterworths, Boston Mass. 1988) and Gilboa et al. (Biotechniques 4 (6): 504-512, 1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods for inducing homologous recombination.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector introducing and expressing recombination sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in may cases, to elude host defense mechanisms. Typically, viruses infect and propagate in specific cell types. The targeting specificity of viral utilizes its natural specificity of viral vectors utilizes its natural specificity to specifically target predetermined cell types and thereby introduce a recombinant gene into the infected cell. The vector to be used according to the invention will depend on desired cell type to be targeted and will be known to those skilled in the art. If diseases or pathological conditions of the blood system are to be treated, then a viral vector that is specific for blood cells and their precursors or liver cells would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment, administration can also be performed by, for example, intravenous or subcutaneous injection into the subject. Injection of the viral vectors into a spinal fluid can also be used as a mode of administration, especially in the case of neuro-degenerative diseases.

Following injection, the viral vectors will circulate until they recognize host cells with appropriate target specificity for infection.

The following patent references describe gene therapy methods and/or protocols used in the formation of gene therapy vehicles and which can be adopted while implementing the present invention:

U.S. Pat. No. 5,683,866, to Sarkar et al., entitled “process for producing a targeted gene”, and which is incorporated by reference as if fully set forth herein, discloses a reconstituted sendai-viral envelope containing the F-protein (F-virosomes) and to a process for producing a targeted gene or drug delivery carrier produced by the steps of chemical reduction of Sendai virus for reduction of HN protein and subjecting the reduced virus to the step of dialysis for removal of the reducing agent. The reduced virus is then solubilized with a detergent to obtain a solution. The said solution is centrifuged to separate the insolubles consisting of reduced HN protein and core of the virus, adding the required specific gene or drug to the centrifugal solution. Finally, the detergent is removed using an affinity complex agent which binds the detergent leading to the formation of the delivery carrier.

U.S. Pat. No. 5,455,027 to Zalipsky et al., entitled “poly(alkylene oxide) amino acid copolymers and drug carriers and charged copolymers based thereon”, and which is incorporated by reference as if fully set forth herein, teaches copolymers of poly(alkylene oxides) and amino acids or polypeptide sequences which have pendant functional groups that are capable of being conjugated with pharmaceutically active compounds for drug delivery systems and cross-linked to form polymer matrices functional as hydrogel membranes. The copolymers can also be formed into conductive materials. Methods are also disclosed for preparing the polymers and forming the drug conjugates, hydrogel membranes and conductive materials.

U.S. Pat. No. 5,652,130 to Kriegler et al., entitled “retroviral vectors expressing tumor necrosis factor (TNF)”, and which is incorporated by reference as if fully set forth herein, discloses a drug delivery virion which contains an expression system for the desired protein active ingredient packaged in an envelope derived from a retrovirus is especially useful in administering materials which need to cross cell membranes in order to serve their function.

U.S. Pat. No. 5,635,399 to Kriegler et al., entitled “retroviral vectors expressing cytokines”, and which is incorporated by reference as if fully set forth herein, similarly teaches a drug delivery virion which contains an expression system for the desired protein active ingredient packaged in an envelope derived from a retrovirus is especially useful in administering materials which need to cross cell membranes in order to serve their function.

U.S. Pat. No. 5,580,575 to Unger et al., entitled “therapeutic drug delivery systems”, and which is incorporated by reference as if fully set forth herein, teaches therapeutic drug delivery systems comprising gas-filled microspheres comprising a therapeutic are described. Methods for employing such microspheres in therapeutic drug delivery applications are also provided. Drug delivery systems comprising gas-filled liposomes having encapsulated therein a drug are preferred. Methods of and apparatus for preparing such liposomes and methods for employing such liposomes in drug delivery applications are also disclosed.

Different designs for gene therapy are also disclosed in Huber E., B. and Magrath I. 1998. Gene therapy in the treatment of cancer. Cambridge University Press., which is incorporated herein by reference.

A compound according to the present invention, either a polypeptide or a nucleic acid, can be administered to an organism, such as a human being or any other mammal, per se, or in a pharmaceutical composition where it is mixed with suitable carriers or excipients.

Thus, according to another aspect of the present invention, there is provided a pharmaceutical composition comprising, as an active ingredient, the antioxidant compound or nucleic acid construct described herein and a pharmaceutically acceptable carrier. Preferably, the pharmaceutical composition is packaged and identified as containing an antioxidant; and/or for use in relieving oxidative stress; and/or for use in a pathology or habit associated with elevated oxidative stress.

As used herein a “pharmaceutical composition” refers to a preparation of one or more of the compounds described herein, or physiologically acceptable salts or prodrugs thereof, with other chemical components such as physiologically suitable carriers and excipients. The purpose of a pharmaceutical composition is to facilitate administration of a compound to an organism.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Pharmaceutical compositions may also include one or more additional active ingredients, such as, but not limited to, anti inflammatory agents, antimicrobial agents, anesthetics and the like in addition to the antioxidant compounds.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the compounds for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Pharmaceutical compositions for parenteral administration include aqueous solutions of the active preparation in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile, pyrogen-free water, before use.

The compounds of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of antioxidant preparation effective to prevent, reduce or alleviate symptoms of disease or prolong the survival of the subject being treated.

Determination of a therapeutically effective amount is well within the capability of those skilled in the art, especially in light of the detailed disclosure provided herein.

Toxicity and therapeutic efficacy of the compounds described herein can be determined by standard pharmaceutical procedures in cell cultures or experimental animals, e.g., by determining the IC₅₀ and the LD₅₀ (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Depending on the severity and responsiveness of the condition to be treated, dosing can also be a single administration of a slow release composition, with course of treatment lasting from several days to several weeks or until cure is effected or diminution of the disease state is achieved. Slow release is effected by trapping the active ingredient of a pharmaceutical composition in, for example, a matrix which forms a solid support, which may form an article or be granulated. the solid support is selected that under physiological conditions it slowly releases the trapped active ingredients. The solid support used in context of this embodiment of the present invention can be a stent.

Stents are expandable prostheses employed to maintain narrow vascular and endoluminal ducts or tracts of the human body open and unoccluded, such as a portion of the lumen of a coronary artery after dilatation of the artery by balloon angioplasty. While vascular usage is frequently discussed in this application, it will be understood by those skilled in the art that stents having the characteristics and features of the present invention may be implanted in other ducts or tracts of the human body to keep the lumen open, such as in the tracheo-bronchial system, the binary hepatic system, the esophageal bowel system, and the urinary tract system. U.S. Pat. Nos. 6,214,868; 5,900,246; 5,693,085; and 5,562,922, for example, each teach coated stents and other bioprostheses designed for the release of drugs, including various antioxidants.

The amount of a composition to be administered will, of course, be dependent on the subject being treated, the severity of the affliction, the manner of administration, the judgment of the prescribing physician, etc.

The present invention can be used to treat any one of a plurality of diseases, disorders or conditions associated with the formation of oxidative stress.

As used herein, the term “treat” include substantially inhibiting, slowing or reversing the progression of a disease, disorder or condition, substantially ameliorating clinical symptoms of a disease disorder or condition, or substantially preventing the appearance of clinical symptoms of a disease, disorder or condition.

The compounds according to the present invention can be used to treat central nervous system neurodegenerative disorders such as, but not limited to, Parkinson's disease, Alzheimer's disease, Creutzfeldt-Jakob's disease, cerebral ischemia, Multiple Sclerosis, basal ganglia degenerative disease, motoneuron diseases, Scrapie, spongyform encephalopathy and loss or impaired memory, peripheral tissue disorders such as, but not limited to, acute respiratory distress syndrome, amyotrophic lateral sclerosis, atherosclerotic cardiovascular disease, multiple organ dysfunction, complication resulting from inflammatory processes, AIDS, cancer, aging and diabetes, all of which were previously shown to be associated with the formation and/or overproduction of oxidants, and habits or medical treatments resulting in oxidative stress, such as, but not limited to, smoking, sun tanning, cancer treatment, exposure to radiation such as radiotherapy, cocaine consumption and morphine consumption.

Hence, according to yet another aspect of the present invention there is provided a method of reducing oxidative stress in a subject in need, the method is effected by administering to the subject an antioxidant compound or a nucleic acid construct as described herein, per se, or as an active ingredient of a pharmaceutical composition that may further include a pharmaceutically acceptable carrier as described herein. The reason for thus treating the individual may be associated with either a pathology and/or a habit and/or medical treatment.

In particular, the present invention can be used to augment the antioxidation capacity of the serum of individuals having haptoglobin type 2, which has poorer antioxidation capabilities as compared to haptoglobin type 1. Most particularly, the present invention can be used to treat diabetic patients in order to prevent macro and microvascular complications associated with prolonged exposure to hyperglycemia, characteristic of diabetes (both type I and type II diabetes), such as, but not limited to, retinopathy, nephropathy, restenosis after angioplasty and atherosclerotic cardiovascular disease.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Experimental Procedures

Oxidation of Linolenic Acid by Hemoglobin:

All reagents were from Sigma Israel (Rehovot) unless otherwise indicated. Fatty acid micelles were prepared by adding 1 μl of linolenic acid to 1 ml of buffer A (50 mM Tris.HCl, pH 6.5) and vortexing vigorously for 10 seconds. Hemoglobin (Sigma H-7379) was prepared at a concentration of 10 mg/ml in buffer A and used within 4 hours of its preparation. Haptoglobin (1-1 or 2-2) was prepared in buffer A and the concentration of the solution determined from the calculated extinction coefficient of haptoglobin (EmM 53.9 for Haptoglobin 1-1 and EmM 58.65 for Haptoglobin 2-2). The molar concentration of haptoglobin was based on the monomer properties of that particular type of haptoglobin. This was done because each haptoglobin monomer (whether in the 1-1 or 2-2 complex) is thought to be capable of binding a single hemoglobin molecule [2]. The standard reaction (720 μl) consisted of the following reagents all incubated at room temperature: 120 μl of the micelles (final concentration of linolenic acid 0.55 mM), 3 μl of a 10 mg/ml solution of hemoglobin in 1 ml of buffer A (final concentration of hemoglobin 0.62 μM), and haptoglobin diluted in buffer A to the desired concentration. Additional buffer A was added to achieve a final volume of 720 μl. All the components except for the hemoglobin were added to a quartz cuvette directly and mixed by inverting 6 times. The 3 μl of the hemoglobin solution was then added and the cuvette inverted to mix the ingredients. The zero time point was designated as the time at which the hemoglobin was added to the solution. The formation of conjugated dienes was monitored by the change in the absorption of the solution at 232 nm (A232) at room temperature for 60 minutes using a WPA Lightwave S2000 spectrophotometer (WPA, Cambridge, UK). Readings were taken every 10 minutes. For all experiments assessing the ability to inhibit diene formation with haptoglobin or vitamins, six simultaneous reactions were performed to permit direct comparison of the increase in A232 obtained from incubation of hemoglobin alone as compared to hemoglobin with the antioxidants to be tested at the various concentrations. The change in A232 with hemoglobin alone at 60 minutes was taken as the 100% value and the change in A232 with each of the antioxidants at the different concentrations of haptoglobin at 60 minutes was determined relative to this value and expressed as a percentage of relative oxidation. For each concentration of each antioxidant the reaction was performed at least 6 separate times and the mean+/−SME determined. P values were determined using paired student's t test with p<0.05 being considered as being statistically significant.

Oxidation of LDL by Hemoglobin:

LDL was isolated from human plasma by sequential ultracentrifugation as previously described [11-12]. Oxy-Hb was obtained by chromatography methods, verified spectrophotometrically and converted to met-Hb as previously described [13]. LDL (200 μg/ml) was incubated for 4 hours at 37° C. with met-Hb (10 μM) in the presence of H₂O₂ (20 μM). To this standard assay were added various concentrations of haptoglobin 1-1 or 2-2 at a range of concentrations from 0.625-20 μM. Oxidation of LDL lipids was determined using the TBARS assay [14] using a WPA lightwave spectrophotometer. All experiments were performed a minimum of three times and the data are presented as mean inhibition by haptoglobin compared to the absence of haptoglobin. The relative inhibition was calculated by integrating the area under the curve of the TBARS assay using MATLAB program. All values are expressed as the mean+/−SME. P values were determined using paired student's t test with p<0.05 being considered as statistically significant.

Preparation of Recombinant Truncated Haptoglobin:

A series of truncated haptoglobin-GST fusion proteins were prepared as follows. Haptoglobin cDNA was prepared from the human Hep G2 cell line by RT-PCR. PCR primers were designed to produce a series of truncated haptoglobin products as noted in Tables 4 and 5 and shown schematically in FIG. 1.

TABLE 4

Primer Sequences Used for Cloning Hp β and o Sub-units constructs

| | | |
|---|---|---|
| Primer name | Sequence | SEQ ID NO: |

| | | |
|---|---|---|
| F-β-1 | 5′-CGCGGATCCATCCTGGGTGGACACCTGGATGCC-3′ | 1 |
| R-β-245 | 5′-GCGGAATTCTTAGTTCTCAGCTATGGTCTTCTGAAC-3′ | 2 |
| F-β80 | 5′-CGCGGATCCAACTACTCCCAGGTAGATATTGGGCTC-3′ | 3 |
| R-β161 | 5′-GCGGAATTCTTACTTCTTTTCGGGGACTGTGCT-3′ | 4 |
| F-β-120 | 5′-CGCGGATTCGTTTCTGGGTGGGGGCGAAATGCC-3′ | 5 |
| R-β-140 | 5′-GCGGAATTCTTACAGCATGACATACTTCAGATG-3′ | 6 |
| F-o-1 | 5′-CGCGGATCCGTAGACTCAGGCAATGATGTCACG-3′ | 7 |
| R-o-70,129 | 5′-GCGGAATTCTATGCTTCACATTCAGGAAGTTT-3′ | 8 |

TABLE 5
Primer Pairs and Deduced Products

| | | | Forward | Reverse | | | |
|---|---|---|---|---|---|---|---|
| | | | Primer | Primer | DNA | Protein | |
| | | | Identified | Identified | fragment | size, amino | Fusion |
| | Hp sub-unit | | by a SEQ | by a SEQ | size, bp | acids | Protein |
| RHp No. | (aa-aa*) | | ID NO: | ID NO: | (SEQ ID NO:) | (SEQ ID NO:) | Size (kDa) |
| RHp1 | β | (1-245) | 1 | 2 | 735 (9) | 245 (15) | 66.0 |
| RHp2 | β | (80-161) | 3 | 4 | 243 (10) | 81 (16) | 39.2 |
| RHp3 | β | (120-161) | 5 | 4 | 123 (11) | 41 (17) | 32.7 |
| RHp4 | β | (120-190) | 5 | 6 | 60 (12) | 20 (18) | 29.3 |
| RHp5 | α | (1-129) | 7 | 8 | 387 (13) | 129 (19) | 42.0 |
| RHp6 | α | (1-70) | 7 | 8 | 210 (14) | 70 (20) | 34.9 |

*aa—amino acid

These PCR products were first cloned into Teasy (Promega), sequenced and then subcloned into pGEX-2TK (Pharmacia). Recombinant protein from pGEX was prepared by induction of logarithmically growing BL21(923) cultures with 0.1 mM IPTG and purification of the sonicated cell lysate on GST-Sepharose (Bio-Rad) as previously described [15]. The GST-haptoglobin fusion protein was analyzed for purity on SDS-PAGE followed by Coomassie blue staining. The concentration of the fusion proteins was determined by the Bradford reagent.

ELISA Assay for Qualitative Determination of Binding of Truncated Haptoglobin to Hemoglobin:

The relative ability of the truncated haptoglobin fusion proteins to bind to hemoglobin was determined in an ELISA assay. A specified amount of purified recombinant haptoglobin (1-250 μg) in TBS (10 mM Tris-buffered saline, pH 8.0) was incubated in a 96-well plate overnight. The haptoglobin solution was then aspirated, washed five times with TBS and blocking then performed with a 5% dry milk solution in TBS for 1 hour. 20 μg of hemoglobin at a concentration of 200 μg/ml in TBS was then added for a one hour incubation at room temperature. The hemoglobin was then aspirated, the plate washed five times with TBS and a monoclonal anti-hemoglobin antibody was then added (Rabbit anti-human hemoglobin, DAKO) at a 1:2000 dilution for overnight incubation at room temperature. This anti-hemoglobin antibody was then removed, the wells washed 5 times with TBS and then incubated with anti-rabbit AP conjugated secondary antibody (Santa Cruz) at a 1:2000 dilution. After again washing the plates 5 times, pNPP (p-Nitrophenyl phosphate, Sigma) was added according to the manufacturer's protocol and the absorbance at 405 nM recorded over time. GST alone or TBS alone were used as negative controls and haptoglobin purified from human serum was used as a positive control. In this qualitative assay, binding was categorized as being none (not significantly different from TBS or GST), 2+ if significant binding was present using less than 10 μg of the recombinant protein and 1+ if significant binding was present only when using greater than 100 μg of recombinant protein in the assay.

Antioxidant Activity of Truncated Haptoglobin:

Recombinant GST-fusion proteins were analyzed in the linolenic acid oxidation assay for their ability to inhibit the oxidation of linolenic acid by hemoglobin as described above. GST alone had no effect on the oxidation of linolenic acid by hemoglobin even when used at concentrations 10 fold greater than that used for the recombinant GST-haptoglobin fusion proteins.

### Experimental Results

Inhibition of Oxidation of Linolenic Acid by Purified Haptoglobin:

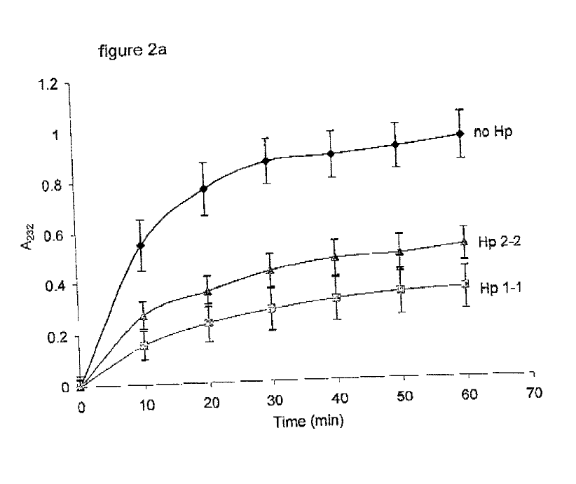

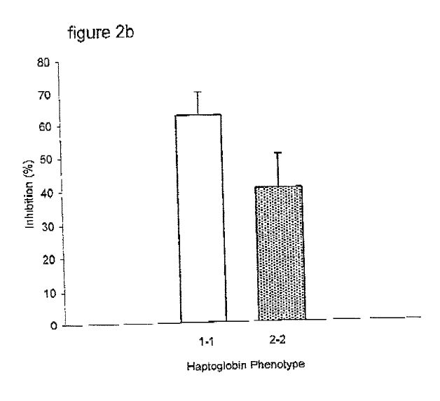

As previously demonstrated, it was found that hemoglobin can oxidize linolenic acid in a time-dependent fashion as assessed using conjugated diene (A232) formation (FIG. 2a). This oxidation of linolenic acid by hemoglobin was previously shown to be inhibited by stoichiometric amounts of a mixture of the different haptoglobins prepared from pooled human sera [10-17]. It was sought to determine if the ability to inhibit the oxidation of linolenic acid by hemoglobin as assessed by diene formation was different between haptoglobin 1-1 and 2-2 proteins. FIG. 2a provides a representative example of the differences in diene formation produced by the oxidation of linolenic acid in the presence of no hemoglobin, haptoglobin 1-1 or haptoglobin 2-2 using 0.6 μM haptoglobin. At this haptoglobin concentration, haptoglobin 1-1 was shown to provide a statistically significant greater protection against linolenic oxidation as compared to haptoglobin 2-2 (FIG. 2b). These differences between haptoglobin 1-1 and 2-2 were maintained over a wide range of haptoglobin concentrations in this assay (0.1-1.0 μM) in which the percent inhibition of oxidation of linolenic acid by haptoglobin was linearly related to haptoglobin concentration.

Inhibition of LDL Oxidation by Haptoglobin:

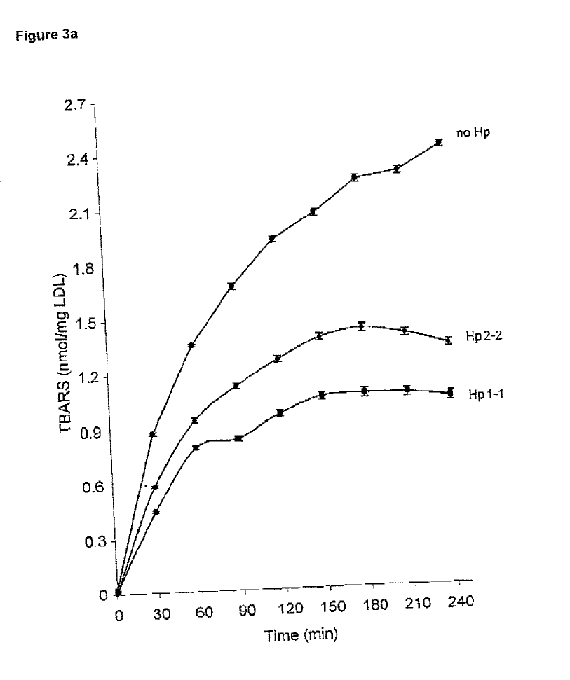

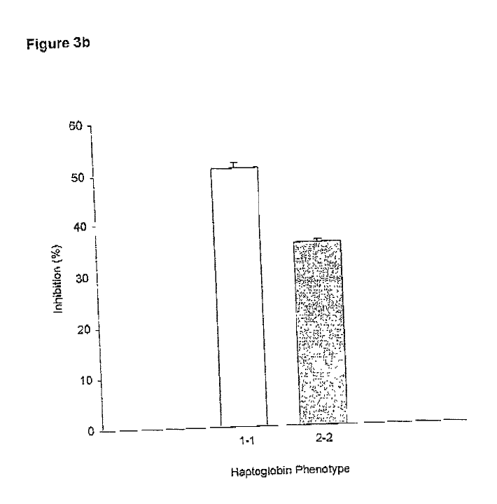

As previously demonstrated [13], it was found that hemoglobin can oxidize LDL in a time-dependent fashion as assessed by measuring TBARS (FIG. 3a). This oxidation of LDL by hemoglobin was previously shown to be inhibited by stoichiometric amounts of a mixture of haptoglobin proteins prepared from pooled human sera [13, 17] It was sought to determine if the ability to inhibit the oxidation of LDL by hemoglobin was different between haptoglobin 1-1 and 2-2 proteins. FIG. 3a provides a representative example of the differences in TBARS formation produced by the oxidation of LDL in the presence of no hemoglobin, haptoglobin 1-1 or haptoglobin 2-2 using 5.0 μM haptoglobin. At this haptoglobin concentration, haptoglobin 1-1 provided a statistically significant greater protection against LDL oxidation as compared to haptoglobin 2-2 (FIG. 3b). These differences between haptoglobin 1-1 and 2-2 were maintained over the range of haptoglobin concentrations in this assay in which the percent inhibition of oxidation of LDL by haptoglobin was linearly related to haptoglobin concentration.

Identification of Putative Hemoglobin Binding Sites on Haptoglobin by ELISA Using Truncated Haptoglobin:

The hemoglobin-haptoglobin complex has not as yet been crystallized and thus the residues involved in binding are not definitively known. Gel permeation studies with purified haptoglobin have suggested that the beta-subunit of haptoglobin is responsible for binding to hemoglobin [1]. The importance of several residues in the beta subunit has been suggested by using proteolytic peptides of haptoglobin and ascertaining their ability to bind to hemoglobin in native polyacrylamide gels [18]. Further assessment of the putative residues on haptoglobin capable of binding to hemoglobin using recombinant haptoglobin truncated mutants or haptoglobin peptides has not been performed. An ELISA assay was therefore developed, capable of differentiating qualitative differences in the binding of haptoglobin to hemoglobin. A battery of alpha and beta subunit recombinant fusion proteins were made and are shown schematically in FIG. 1. The inventors were able to identify binding not only in the beta subunit but surprisingly also in the alpha subunit (qualitatively denoted by 0-2+ binding in Table 6).

TABLE 6
Binding of Truncated Haptoglobin to Hemoglobin

| Substrate | |
|---|---|
| (SEQ ID NO:) | Binding |
| TBS | − |
| GST | − |
| RHp1 (15) | ++ |
| RHp2 (16) | ++ |
| RHp3 (17) | + |
| RHp4 (18) | − |
| RHp5 (19) | + |
| RHp6 (20) | ++ |

Truncated Haptoglobin can Prevent the Oxidation of Linolenic Acid by Hemoglobin:

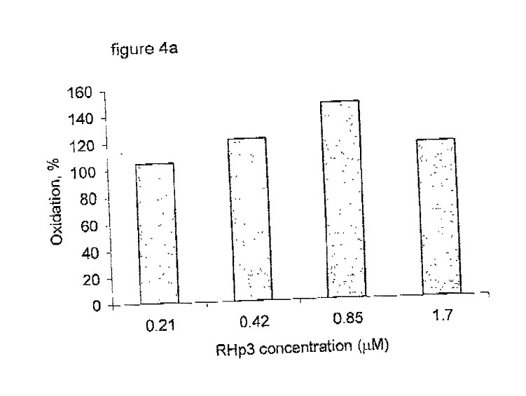

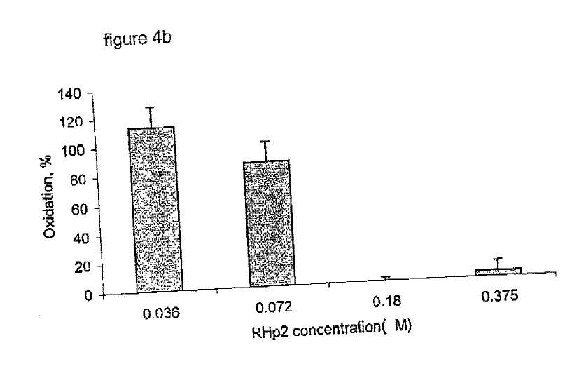

The truncated haptoglobin fusion proteins that were shown to bind to hemoglobin in the ELISA assay were tested for their ability to inhibit the oxidation of linolenic acid by hemoglobin as described in the Experimental Procedures section above. Using progressive deletion analysis a 81 amino acid fragment (construct RHp2, Table 4, SEQ ID NO:16) of the beta subunit was identified, that could completely prevent the oxidation of linolenic acid by hemoglobin at concentrations similar to what were seen with native haptoglobin (FIG. 4a). A 41 amino acid fragment (SEQ ID NO:17) within this 81 amino acid fragment (SEQ ID NO:16) could not inhibit the oxidation of linolenic acid even though it demonstrated specific binding to hemoglobin (FIG. 4b, Table 6).

It was also demonstrated that the alpha subunit of haptoglobin (RHp 5 or 6, corresponding to the alpha subunit from haptoglobin allele 1 or 2, SEQ ID NOs:19 and 20) can inhibit oxidation by hemoglobin as efficiently as haptoglobin.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their genebank accession numbers mentioned in this specification are herein incorporated in their entirety by reference into the specification, to the same extent as if each individual publication, patent, patent application or sequence was specifically and individually indicated to be incorporated herein by reference. In addition, citation or identification of any reference in this application shall not be construed as an admission that such reference is available as prior art to the present invention.

### REFERENCES CITED

### Additional References are Cited in the Text

1. Bowman B H, Kurosky A. Haptoglobin: the evolutionary product of duplication, unequal crossing over, and point mutation. Adv Hum Gen 1982; 12: 189-261.

2. Langlois M R, Delanghe J R. Biological and clinical significance of haptoglobin polymorphism in humans. Clin Chem 1996; 42: 1589-1600.

3. Lim S K, Kim H, Lim S K, et al. Increased susceptibility in Hp knockout mice during acute hemolysis. Blood 1998; 92: 1870-1877.

4. Dobryszycka W. Biological functions of haptoglobin-new pieces to an old puzzle. Eur J Clin Chem 1997; 35: 647-654.

5. Levy A P, Roguin A, Marsh S, et al. Haptoglobin phenotype and vascular complications in diabetes. N Eng J Med 2000; 343: 369-370.

6. Nakhoul F, Marsh S, Hochberg I, Leibu R, Miller B, Levy A P. Haptoglobin phenotype and diabetic retinopathy. JAMA 2000; 284: 1244-1245.

7. Nakhoul F, Zoabi R, Kantor Y, et al. Haptoglobin phentotype and diabetic nephropathy. Diabetologia 2001; 44: 602-604.

8. Roguin A, Hochberg I, Nikolsky E, et al. Haptoglobin phenotype as a predictor of restenosis after percutaneous transluminal coronary angioplasty. Am J Card 2001; 87: 330-332.

9. Guigliano D, Ceriello A, Paolisso G. Oxidative stress and diabetic vascular complications. Diab Care 1996; 19: 257-267.

10. Nishikawa T, Edelstein D, Brownlee M. The missing link: a single unifying mechanism for diabetic complications. Kid Int 2000; 58 (S77): S26-S30.

11. Schumaker V N, Puppione D L. Sequential flotation ultracentrifugation. Meth Enz 1986; 128: 155-169.

12. Miller Y I, Felikman Y, Shaklai N. Hemoglobin induced apolipoprotein B cross-linking in low-density lipoprotein peroxidation. Arch Biochem Biophys 1996; 326: 252-260.

13. Miller Y I, Altamentova S M, Shaklai N. Oxidation of low-density lipoprotein by hemoglobin stems from a heme-initiated globin radical: antioxidant role of haptoglobin. Biochemistry 1997; 36: 12189-12198.

14. Balla G, Jacob H S, Eaton J W, Belcher J D, Vercellotti G M. Hemin: a possible physiological mediator of low density lipoprotein oxidation and endothelial injury. Arterioscler Thromb 1991; 11: 1700-1711.

15. Levy N S, Chang S, Furneaux H M, Levy A P. Hypoxic stabilization of VEGF mRNA by the RNA-binding protein HuR. J Biol Chem 1998; 273: 6417-6423.

16. Gutteridge J M. The antioxidant activity of haptoglobin towards hemoglobin-stimulated lipid peroxidation. Biochim Biophys Acta 1987; 917: 219-223.

17. Toda S, Ohnishi M, Kimura M et al. Peroxidation of linolenic acid induced by interaction with hemoglobin and hydrogen peroxide. Free Rad Res Comm 1989; 6: 203-208.

18. Lustbader J W, Arcoleo J P, Birken S, Greer J. Hemoglobin-binding site on haptolglobin probed by selective proteolysis. J Biol Chem 1983; 258:1227-1234.

19. Langlois M R, Delanghe J R, De Buyzere M L, Bernard D R, Ouyang J. Effect of haptoglobin on the metabolism of vitamin C. Am J Clin Nutr 1997; 66; 606-610.

20. Wejman J C, Hovsepian D, Wall J S, Hainfeld J F, Greer J. Structure and assembly of haptoglobin polymers by electron microscopy. J Mol Biol 1984; 174: 343-368.

21. Kristiansen M, Graversen J H, Jacobsen C, et al. Identification of the hemoglobin scavenger receptor. Nature 2001; 409: 198-201.

22. Langlois M R, Martin M E, Boelaert J R et al. The haptoglobin 2-2 phenotype affects serum markers of iron status in healthy males. Clin Chem 2000; 46: 1619-1625.

What is claimed is:

1. A method of evaluating a potential of a haptoglobin derived polypeptide in reducing oxidation induced by oxygenized hemoglobin, the method comprising:
reacting hemoglobin with an oxidizable substrate in a presence and an absence, and/or in a presence of varying concentrations of the haptoglobin derived polypeptide; and
determining an effect of said presence and said absence, and/or said presence of said varying concentrations of the haptoglobin derived polypeptide on oxidation of said oxidizable substrate, thereby evaluating the potential of the haptoglobin derived polypeptide in reducing the oxidation induced by the hemoglobin.

2. The method of claim 1, wherein said oxidizable substrate comprises a fatty acid.

3. The method of claim 1, wherein said oxidizable substrate comprises an unsaturated fatty acid.

4. The method of claim 1, wherein said oxidizable substrate comprises low density lipoprotein (LDL).

5. The method of claim 1, wherein said oxidizable substrate comprises very low density lipoprotein (VLDL).

6. The method of claim 1, wherein said oxidizable substrate comprises chylomicrons.

7. The method of claim 1, wherein determining said effect is by monitoring at least one oxidation product of said oxidizeable substrate.

8. The method of claim 7, wherein said oxidation product comprises conjugated dienes.

9. The method of claim 7, wherein monitoring said at least one oxidation product of said oxidizeable substrate is effected spectrally.

10. An antioxidant compound comprising a polypeptide having an amino acid sequence derived from an alpha subunit of a haptoglobin protein sequence, said polypeptide being capable of reducing oxidation induced by oxygenized hemoglobin, the antioxidant compound being free of amino acid sequences derived from a beta subunit of a haptoglobin protein.

11. The antioxidant of claim 10, wherein said haptoglobin protein sequence is of a mammal.

12. The antioxidant of claim 11, wherein said mammal is selected from the group consisting of human, mouse, rat and dog.

13. The antioxidant of claim 11, wherein said mammal is human.

14. The antioxidant of claim 10, wherein said polypeptide has an amino acid sequence derived from a portion of an alpha subunit of a haptoglobin protein sequence, said portion being capable of reducing oxidation induced by oxygenized hemoglobin.

15. The antioxidant of claim 10, wherein said polypeptide is as set forth in SEQ ID NO:19.

16. The antioxidant of claim 10, wherein said polypeptide is as set forth in SEQ ID NO:20.

17. A pharmaceutical composition comprising, as an active ingredient, the antioxidant of claims 10-16, and a pharmaceutically acceptable carrier.

18. The pharmaceutical composition of claim 17, packaged and identified as containing an antioxidant.

19. The pharmaceutical composition of claim 17, packaged and identified for use in relieving oxidative stress.

20. The pharmaceutical composition of claim 17, packaged and identified for use in a pathology or habit associated with elevated oxidative stress.

21. The pharmaceutical composition of claim 17, wherein said pharmaceutically acceptable carrier comprises a solid support.

22. The pharmaceutical composition of claim 21, wherein said solid support is a stent.

23. The pharmaceutical composition of claim 17, wherein said pharmaceutically acceptable carrier is designed for slow release.

24. An antioxidant compound comprising a polypeptide having an amino acid sequence derived from a portion of an alpha subunit of a haptoglobin protein sequence, said polypeptide being capable of reducing oxidation induced by oxygenized hemoglobin, said polypeptide being free of remaining portions of said alpha subunit of said haptoglobin protein sequence.

25. The antioxidant of claim 24, wherein said haptoglobin protein sequence is of a mammal.

26. The antioxidant of claim 25, wherein said mammal is selected from the group consisting of human, mouse, rat and dog.

27. The antioxidant of claim 25, wherein said mammal is human.

28. The antioxidant of claim 24, wherein said polypeptide has an amino acid sequence derived from a consecutive portion of said alpha subunit of a haptoglobin protein sequence, said portion being capable of reducing oxidation induced by oxygenized hemoglobin.

29. The antioxidant of claim 24, wherein said polypeptide is as set forth in SEQ ID NO:19.

30. The antioxidant of claim 24, wherein said polypeptide is as set forth in SEQ ID NO:20.

31. A pharmaceutical composition comprising, as an active ingredient, the antioxidant compound of any of claims 24-30, and a pharmaceutically acceptable carrier.

32. The pharmaceutical composition of claim 31, packaged and identified as containing an antioxidant.

33. The pharmaceutical composition of claim 31, packaged and identified for use in relieving oxidative stress.

34. The pharmaceutical composition of claim 31, packaged and identified for use in a pathology or habit associated with elevated oxidative stress.

35. The pharmaceutical composition of claim 31, wherein said pharmaceutically acceptable carrier comprises a solid support.

36. The pharmaceutical composition of claim 35, wherein said solid support is a stent.

37. The pharmaceutical composition of claim 31, wherein said pharmaceutically acceptable carrier is designed for slow release.

38. An antioxidant compound comprising a polypeptide having an amino acid sequence derived from a beta subunit of a haptoglobin protein sequence, said polypeptide being capable of reducing oxidation induced by oxygenized hemoglobin, the antioxidant compound being free of amino acid sequences derived from an alpha subunit of a haptoglobin protein.

39. The antioxidant of claim 38, wherein said haptoglobin protein sequence is of a mammal.

40. The antioxidant of claim 39, wherein said mammal is selected from the group consisting of human, mouse, rat and dog.

41. The antioxidant of claim 39, wherein said mammal is human.

42. The antioxidant of claim 38, wherein said polypeptide has an amino acid sequence derived from a portion of said beta subunit of a haptoglobin protein sequence, said portion being capable of reducing oxidation induced by oxygenized hemoglobin.

43. The antioxidant of claim 38, wherein said polypeptide is as set forth in SEQ ID NO:15.

44. The antioxidant of claim 38, wherein said polypeptide is as set forth in SEQ ID NO:16.

45. A pharmaceutical composition comprising, as an active ingredient, the antioxidant compound of any of claims 38-44, and a pharmaceutically acceptable carrier.

46. The pharmaceutical composition of claim 45, packaged and identified as containing an antioxidant.

47. The pharmaceutical composition of claim 45, packaged and identified for use in relieving oxidative stress.

48. The pharmaceutical composition of claim 45, packaged and identified for use in a pathology or habit associated with elevated oxidative stress.

49. The pharmaceutical composition of claim 45, wherein said pharmaceutically acceptable carrier comprises a solid support.

50. The pharmaceutical composition of claim 49, wherein said solid support is a stent.

51. The pharmaceutical composition of claim 45, wherein said pharmaceutically acceptable carrier is designed for slow release.

52. An antioxidant compound comprising a polypeptide having an amino acid sequence derived from a portion of a beta subunit of a haptoglobin protein sequence, said polypeptide being capable of reducing oxidation induced by oxygenized hemoglobin, said polypeptide being free of remaining portions of said beta subunit of a haptoglobin protein sequence.

53. The antioxidant of claim 52, wherein said haptoglobin protein sequence is of a mammal.

54. The antioxidant of claim 53, wherein said mammal is selected from the group consisting of human, mouse, rat and dog.

55. The antioxidant of claim 53, wherein said mammal is human.

56. The antioxidant of claim 52, wherein said polypeptide has an amino acid sequence derived from a consecutive portion of a beta subunit of a haptoglobin protein sequence, said portion being capable of reducing oxidation induced by oxygenized hemoglobin.

57. The antioxidant of claim 52, wherein said polypeptide is as set forth in SEQ ID NO:15.

58. The antioxidant of claim 52, wherein said polypeptide is as set forth in SEQ ID NO:16.

59. A pharmaceutical composition comprising, as an active ingredient, the antioxidant compound of any of claims 52-58, and a pharmaceutically acceptable carrier.

60. The pharmaceutical composition of claim 59, packaged and identified as containing an antioxidant.

61. The pharmaceutical composition of claim 59, packaged and identified for use in relieving oxidative stress.

62. The pharmaceutical composition of claim 59, packaged and identified for use in a pathology or habit associated with elevated oxidative stress.

63. The pharmaceutical composition of claim 59, wherein said pharmaceutically acceptable carrier comprises a solid support.

64. The pharmaceutical composition of claim 63, wherein said solid support is a stent.

65. The pharmaceutical composition of claim 59, wherein said pharmaceutically acceptable carrier is designed for slow release.

66. A method of reducing oxidative stress in a subject in need, the method comprising administering to the subject an antioxidant compound that comprises a polypeptide having an amino acid sequence derived from an alpha subunit of a haptoglobin protein sequence, said polypeptide being capable of reducing oxidation induced by oxygenized hemoglobin, the antioxidant compound being free of amino acid sequences derived from a beta subunit of a haptoglobin protein.

67. The method of claim 66, wherein said haptoglobin protein sequence is of a mammal.

68. The method of claim 67, wherein said mammal is selected from the group consisting of human, mouse, rat and dog.

69. The method of claim 67, wherein said mammal is human.

70. The method of claim 66, wherein said polypeptide has an amino acid sequence derived from a portion of an alpha subunit of a haptoglobin protein sequence, said portion being capable of reducing oxidation induced by oxygenized hemoglobin.

71. The method of claim 66, wherein said polypeptide is as set forth in SEQ ID NO:19.

72. The method of claim 66, wherein said polypeptide is as set forth in SEQ ID NO:20.

73. The method of any of claims 66-72, wherein said polypeptide is administered as an active ingredient of a pharmaceutical composition, said pharmaceutical composition further comprises a pharmaceutically acceptable carrier.

74. The method of any of claims 66-72, wherein said oxidative stress is associated with a pathology or habit.

75. The method of claim 73, wherein said pharmaceutically acceptable carrier comprises a solid support.

76. The method of claim 75, wherein said solid support is a stent.

77. The method of claim 73, wherein said pharmaceutically acceptable carrier is designed for slow release.

78. A method of reducing oxidative stress in a subject in need, the method comprising administering to the subject an antioxidant compound that comprises a polypeptide having an amino acid sequence derived from a portion of an alpha subunit of a haptoglobin protein sequence, said polypeptide being capable of reducing oxidation induced by oxygenized hemoglobin, said polypeptide being free of remaining portions of said alpha subunit of said haptoglobin protein sequence.

79. The method of claim 78, wherein said haptoglobin protein sequence is of a mammal.

80. The method of claim 79, wherein said mammal is selected from the group consisting of human, mouse, rat and dog.

81. The method of claim 79, wherein said mammal is human.

82. The method of claim 78, wherein said polypeptide has an amino acid sequence derived from a consecutive portion of said alpha subunit of a haptoglobin protein sequence, said portion being capable of reducing oxidation induced by oxygenized hemoglobin.

83. The method of claim 78, wherein said polypeptide is as set forth in SEQ ID NO:19.

84. The method of claim 78, wherein said polypeptide is as set forth in SEQ ID NO:20.

85. The method of any of claims 78-84, wherein said polypeptide is administered as an active ingredient of a pharmaceutical composition, said pharmaceutical composition further comprises a pharmaceutically acceptable carrier.

86. The method of any of claims 78-84, wherein said oxidative stress is associated with a pathology or habit.

87. The method of claim 85, wherein said pharmaceutically acceptable carrier comprises a solid support.

88. The method of claim 87, wherein said solid support is a stent.

89. The method of claim 87, wherein said pharmaceutically acceptable carrier is designed for slow release.

90. A method of reducing oxidative stress in a subject in need, the method comprising administering to the subject an antioxidant compound that comprises a polypeptide having an amino acid sequence derived from a beta subunit of a haptoglobin protein sequence, said polypeptide being capable of reducing oxidation induced by oxygenized hemoglobin, the antioxidant compound being free of amino acid sequences derived from an alpha subunit of a haptoglobin protein.

91. The method of claim 90, wherein said haptoglobin protein sequence is of a mammal.

92. The method of claim 91, wherein said mammal is selected from the group consisting of human, mouse, rat and dog.

93. The method of claim 91, wherein said mammal is human.

94. The method of claim 90, wherein said polypeptide has an amino acid sequence derived from a portion of said beta subunit of a haptoglobin protein sequence, said portion being capable of reducing oxidation induced by oxygenized hemoglobin.

95. The method of claim 90, wherein said polypeptide is as set forth in SEQ ID NO:15.

96. The method of claim 90, wherein said polypeptide is as set forth in SEQ ID NO:16.

97. The method of any of claims 90-96, wherein said polypeptide is administered as an active ingredient of a pharmaceutical composition, said pharmaceutical composition further comprises a pharmaceutically acceptable carrier.

98. The method of any of claims 90-96, wherein said oxidative stress is associated with a pathology or habit.

99. The method of claim 97, wherein said pharmaceutically acceptable carrier comprises a solid support.

100. The method of claim 99, wherein said solid support is a stent.

101. The method of claim 99, wherein said pharmaceutically acceptable carrier is designed for slow release.

102. A method of reducing oxidative stress in a subject in need, the method comprising administering to the subject an antioxidant compound that comprises a polypeptide having an amino acid sequence derived from a portion of a beta subunit of a haptoglobin protein sequence, said polypeptide being capable of reducing oxidation induced by oxygenized hemoglobin, said polypeptide being free of remaining portions of said beta subunit of a haptoglobin protein sequence.

103. The method of claim 102, wherein said haptoglobin protein sequence is of a mammal.

104. The method of claim 103, wherein said mammal is selected from the group consisting of human, mouse, rat and dog.

105. The method of claim 103, wherein said mammal is human.

106. The method of claim 102, wherein said polypeptide has an amino acid sequence derived from a consecutive portion of a beta subunit of a haptoglobin protein sequence, said portion being capable of reducing oxidation induced by oxygenized hemoglobin.

107. The method of claim 102, wherein said polypeptide is as set forth in SEQ ID NO:15.

108. The method of claim 102, wherein said polypeptide is as set forth in SEQ ID NO:16.

109. The method of any of claims 102-108, wherein said polypeptide is administered as an active ingredient of a pharmaceutical composition, said pharmaceutical composition further comprises a pharmaceutically acceptable carrier.

110. The method of any of claims 102-108, wherein said oxidative stress is associated with a pathology or habit.

111. The method of claim 109, wherein said pharmaceutically acceptable carrier comprises a solid support.

112. The method of claim 111, wherein said solid support is a stent.

113. The method of claim 111, wherein said pharmaceutically acceptable carrier is designed for slow release.

114. A nucleic acid construct comprising:
a first polynucleotide encoding a polypeptide having an amino acid sequence derived from an alpha subunit of a haptoglobin protein sequence, said polypeptide being capable of reducing oxidation induced by oxygenized hemoglobin, the polynucleotide being free of amino acid sequences derived from a beta subunit of a haptoglobin protein; and
a second polynucleotide harboring a promoter operably linked to said first polynucleotide.

115. The nucleic acid construct of claim 114, wherein said haptoglobin protein sequence is of a mammal.

116. The nucleic acid construct of claim 115, wherein said mammal is selected from the group consisting of human, mouse and rat.

117. The nucleic acid construct of claim 115, wherein said mammal is human.

118. The nucleic acid construct of claim 114, wherein said polypeptide has an amino acid sequence derived from a portion of an alpha subunit of a haptoglobin protein sequence, said portion being capable of reducing oxidation induced by oxygenized hemoglobin.

119. The nucleic acid construct of claim 114, wherein said polypeptide is as set forth in SEQ ID NO:19.

120. The nucleic acid construct of claim 114, wherein said first polynucleotide is as set forth in SEQ ID NO:13.

121. The nucleic acid construct of claim 114, wherein said polypeptide is as set forth in SEQ ID NO:20.

122. The nucleic acid construct of claim 114, wherein said first polynucleotide is as set forth in SEQ ID NO:14.

123. A pharmaceutical composition comprising, as an active ingredient, the nucleic acid construct of claims 114-121, and a pharmaceutically acceptable carrier.

124. The pharmaceutical composition of claim 123, packaged and identified for use in relieving oxidative stress.

125. The pharmaceutical composition of claim 123, packaged and identified for use in a pathology or habit associated with elevated oxidative stress.

126. A nucleic acid construct comprising:
a first polynucleotide encoding a polypeptide having an amino acid sequence derived from a portion of an alpha subunit of a haptoglobin protein sequence, said polypeptide being capable of reducing oxidation induced by oxygenized hemoglobin, said polypeptide being free of remaining portions of said alpha subunit of said haptoglobin protein sequence; and
a second polynucleotide harboring a promoter operably linked to said first polynucleotide.

127. The nucleic acid construct of claim 126, wherein said haptoglobin protein sequence is of a mammal.

128. The nucleic acid construct of claim 127, wherein said mammal is selected from the group consisting of human, mouse and rat.

129. The nucleic acid construct of claim 127, wherein said mammal is human.

130. The nucleic acid construct of claim 126, wherein said polypeptide has an amino acid sequence derived from a consecutive portion of said alpha subunit of a haptoglobin protein sequence, said portion being capable of reducing oxidation induced by oxygenized hemoglobin.

131. The nucleic acid construct of claim 126, wherein said polypeptide is as set forth in SEQ ID NO:19.

132. The nucleic acid construct of claim 126, wherein said first polynucleotide is as set forth in SEQ ID NO:13.

133. The nucleic acid construct of claim 126, wherein said polypeptide is as set forth in SEQ ID NO:20.

134. The nucleic acid construct of claim 126, wherein said first polynucleotide is as set forth in SEQ ID NO:14.

135. A pharmaceutical composition comprising, as an active ingredient, the nucleic acid construct of any of claims 126-133, and a pharmaceutically acceptable carrier.

136. The pharmaceutical composition of claim 135, packaged and identified for use in relieving oxidative stress.

137. The pharmaceutical composition of claim 135, packaged and identified for use in a pathology or habit associated with elevated oxidative stress.

138. A nucleic acid construct comprising:
a first polynucleotide encoding a polypeptide having an amino acid sequence derived from a beta subunit of a haptoglobin protein sequence, said polypeptide being capable of reducing oxidation induced by oxygenized hemoglobin, the antioxidant compound being free of amino acid sequences derived from an alpha subunit of a haptoglobin protein; and
a second polynucleotide harboring a promoter operably linked to said first polynucleotide.

139. The nucleic acid construct of claim 138, wherein said haptoglobin protein sequence is of a mammal.

140. The nucleic acid construct of claim 139, wherein said mammal is selected from the group consisting of human, mouse and rat.

141. The nucleic acid construct of claim 139, wherein said mammal is human.

142. The nucleic acid construct of claim 138, wherein said polypeptide has an amino acid sequence derived from a portion of said beta subunit of a haptoglobin protein sequence, said portion being capable of reducing oxidation induced by oxygenized hemoglobin.

143. The nucleic acid construct of claim 138, wherein said polypeptide is as set forth in SEQ ID NO:15.

144. The nucleic acid construct of claim 138, wherein said first polynucleotide is as set forth in SEQ ID NO:9.

145. The nucleic acid construct of claim 138, wherein said polypeptide is as set forth in SEQ ID NO:16.

146. The nucleic acid construct of claim 138, wherein said first polynucleotide is as set forth in SEQ ID NO:10.

147. A pharmaceutical composition comprising, as an active ingredient, the nucleic acid construct of any of claims 138-145, and a pharmaceutically acceptable carrier.

148. The pharmaceutical composition of claim 147, packaged and identified for use in relieving oxidative stress.

149. The pharmaceutical composition of claim 147, packaged and identified for use in a pathology or habit associated with elevated oxidative stress.

150. A nucleic acid construct comprising:
a first polynucleotide encoding a polypeptide having an amino acid sequence derived from a portion of a beta subunit of a haptoglobin protein sequence, said polypeptide being capable of reducing oxidation induced by oxygenized hemoglobin, said polypeptide being free of remaining portions of said beta subunit of a haptoglobin protein sequence; and
a second polynucleotide harboring a promoter operably linked to said first polynucleotide.

151. The nucleic acid construct of claim 150, wherein said haptoglobin protein sequence is of a mammal.

152. The nucleic acid construct of claim 151, wherein said mammal is selected from the group consisting of human, mouse and rat.

153. The nucleic acid construct of claim 151, wherein said mammal is human.

154. The nucleic acid construct of claim 150, wherein said polypeptide has an amino acid sequence derived from a consecutive portion of a beta subunit of a haptoglobin protein sequence, said portion being capable of reducing oxidation induced by oxygenized hemoglobin.

155. The nucleic acid construct of claim 150, wherein said polypeptide is as set forth in SEQ ID NO:15.

156. The nucleic acid construct of claim 150, wherein said first polynucleotide is as set forth in SEQ ID NO:9.

157. The nucleic acid construct of claim 150, wherein said polypeptide is as set forth in SEQ ID NO:16.

158. The nucleic acid construct of claim 150, wherein said first polynucleotide is as set forth in SEQ ID NO:10.

159. A pharmaceutical composition comprising, as an active ingredient, the nucleic acid construct of any of claims 150-157, and a pharmaceutically acceptable carrier.

160. The pharmaceutical composition of claim 159, packaged and identified for use in relieving oxidative stress.

161. The pharmaceutical composition of claim 159, packaged and identified for use in a pathology or habit associated with elevated oxidative stress.

162. A method of reducing oxidative stress in a subject in need thereof, the method comprising administering to the subject a nucleic acid construct that comprises:
a first polynucleotide encoding a polypeptide having an amino acid sequence derived from an alpha subunit of a haptoglobin protein sequence, said polypeptide being capable of reducing oxidation induced by oxygenized hemoglobin, the polynucleotide being free of amino acid sequences derived from a beta subunit of a haptoglobin protein; and
a second polynucleotide harboring a promoter operably linked to said first polynucleotide, so as to direct expression of said polypeptide by at least one cell type of the subject.

163. The method of claim 162, wherein said haptoglobin protein sequence is of a mammal.

164. The method of claim 163, wherein said mammal is selected from the group consisting of human, mouse and rat.

165. The method of claim 163, wherein said mammal is human.

166. The method of claim 162, wherein said polypeptide has an amino acid sequence derived from a portion of an alpha subunit of a haptoglobin protein sequence, said portion being capable of reducing oxidation induced by oxygenized hemoglobin.

167. The method of claim 162, wherein said polypeptide is as set forth in SEQ ID NO:19.

168. The method of claim 162, wherein said first polynucleotide is as set forth in SEQ ID NO:13.

169. The method of claim 162, wherein said polypeptide is as set forth in SEQ ID NO:20.

170. The method of claim 162, wherein said first polynucleotide is as set forth in SEQ ID NO:14.

171. A method of reducing oxidative stress in a subject in need thereof, the method comprising administering to the subject a nucleic acid construct that comprises:
a first polynucleotide encoding a polypeptide having an amino acid sequence derived from a portion of an alpha subunit of a haptoglobin protein sequence, said polypeptide being capable of reducing oxidation induced by oxygenized hemoglobin, said polypeptide being free of remaining portions of said alpha subunit of said haptoglobin protein sequence; and
a second polynucleotide harboring a promoter operably linked to said first polynucleotide, so as to direct expression of said polypeptide by at least one cell type of the subject.

172. The method of claim 171, wherein said haptoglobin protein sequence is of a mammal.

173. The method of claim 172, wherein said mammal is selected from the group consisting of human, mouse and rat.

174. The method of claim 172, wherein said mammal is human.

175. The method of claim 171, wherein said polypeptide has an amino acid sequence derived from a consecutive portion of said alpha subunit of a haptoglobin protein sequence, said portion being capable of reducing oxidation induced by oxygenized hemoglobin.

176. The method of claim 171, wherein said polypeptide is as set forth in SEQ ID NO:19.

177. The method of claim 171, wherein said first polynucleotide is as set forth in SEQ ID NO:13.

178. The method of claim 171, wherein said polypeptide is as set forth in SEQ ID NO:20.

179. The method of claim 171, wherein said first polynucleotide is as set forth in SEQ ID NO:14.

180. A method of reducing oxidative stress in a subject in need thereof, the method comprising administering to the subject a nucleic acid construct that comprises:
a first polynucleotide encoding a polypeptide having an amino acid sequence derived from a beta subunit of a haptoglobin protein sequence, said polypeptide being capable of reducing oxidation induced by oxygenized hemoglobin, the antioxidant compound being free of amino acid sequences derived from an alpha subunit of a haptoglobin protein; and
a second polynucleotide harboring a promoter operably linked to said first polynucleotide, so as to direct expression of said polypeptide by at least one cell type of the subject.

181. The method of claim 180, wherein said haptoglobin protein sequence is of a mammal.

182. The method of claim 181, wherein said mammal is selected from the group consisting of human, mouse and rat.

183. The method of claim 181, wherein said mammal is human.

184. The method of claim 180, wherein said polypeptide has an amino acid sequence derived from a portion of said beta subunit of a haptoglobin protein sequence, said portion being capable of reducing oxidation induced by oxygenized hemoglobin.

185. The method of claim 180, wherein said polypeptide is as set forth in SEQ ID NO:15.

186. The method of claim 180, wherein said first polynucleotide is as set forth in SEQ ID NO:9.

187. The method of claim 180, wherein said polypeptide is as set forth in SEQ ID NO:16.

188. The method of claim 180, wherein said first polynucleotide is as set forth in SEQ ID NO:10.

189. A method of reducing oxidative stress in a subject in need thereof, the method comprising administering to the subject a nucleic acid construct that comprises:
a first polynucleotide encoding a polypeptide having an amino acid sequence derived from a portion of a beta subunit of a haptoglobin protein sequence, said polypeptide being capable of reducing oxidation induced by oxygenized hemoglobin, said polypeptide being free of remaining portions of said beta subunit of a haptoglobin protein sequence; and
a second polynucleotide harboring a promoter operably linked to said first polynucleotide, so as to direct expression of said polypeptide by at least one cell type of the subject.

190. The method of claim 189, wherein said haptoglobin protein sequence is of a mammal.

191. The method of claim 190, wherein said mammal is selected from the group consisting of human, mouse and rat.

192. The method of claim 190, wherein said mammal is human.

193. The method of claim 189, wherein said polypeptide has an amino acid sequence derived from a consecutive portion of a beta subunit of a haptoglobin protein sequence, said portion being capable of reducing oxidation induced by oxygenized hemoglobin.

194. The method of claim 189, wherein said polypeptide is as set forth in SEQ ID NO:15.

195. The method of claim 189, wherein said first polynucleotide is as set forth in SEQ ID NO:9.

196. The method of claim 189, wherein said polypeptide is as set forth in SEQ ID NO:16.

197. The method of claim 189, wherein said first polynucleotide is as set forth in SEQ ID NO:10.


SEQUENCE LISTING (20 sequences)

SEQ ID NO: 1 - DNA, 33 bp, Artificial sequence
  synthetic oligonucleotide
cgcggatccatcctgggtggacacctggatgcc

SEQ ID NO: 2 - DNA, 36 bp, Artificial sequence
  synthetic oligonucleotide
gcggaattcttagttctcagctatggtcttctgaac

SEQ ID NO: 3 - DNA, 36 bp, Artificial sequence
  synthetic oligonucleotide
cgcggatccaactactcccaggtagatattgggctc

SEQ ID NO: 4 - DNA, 33 bp, Artificial sequence
  synthetic oligonucleotide
gcggaattcttacttcttttcggggactgtgct

SEQ ID NO: 5 - DNA, 33 bp, Artificial sequence
  synthetic oligonucleotide
cgcggattcgtttctgggtgggggcgaaatgcc

SEQ ID NO: 6 - DNA, 33 bp, Artificial sequence
  synthetic oligonucleotide
gcggaattcttacagcatgacatacttcagatg

SEQ ID NO: 7 - DNA, 33 bp, Artificial sequence
  synthetic oligonucleotide
cgcggatccgtagactcaggcaatgatgtcacg

SEQ ID NO: 8 - DNA, 33 bp, Artificial sequence
  synthetic oligonucleotide
gcggaattcttatgcttcacattcaggaagttt

SEQ ID NO: 9 - DNA, 738 bp, Homo sapiens
atcctgggtggacaccttgatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctgggtgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggagaacacctggtatgcgactgggatcttaagctttgataagagctgtgctgtggctgagtatggtgtgtatgtgaaggtgacttccatccaagactgggttcagaagaccatagctgagaactaa

SEQ ID NO: 10 - DNA, 246 bp, Homo sapiens
aactactcccaggtagatattgggctcatcaaactcaaacagaaggtgtctgttaatgagagagtgatgcccatctgcctaccttcaaaggattatgcagaagtagggcgtgtgggttatgtttctgggtgggggcgaaatgccaattttaaatttactgaccatctgaagtatgtcatgctgcctgtggctgaccaagaccaatgcataaggcattatgaaggcagcacagtccccgaaaagaag

SEQ ID NO: 11 - DNA, 126 bp, Homo sapiens
gtttctgggtgggggcgaaatgccaattttaaatttactgaccatctgaagtatgtcatgctgcctgtggctgaccaagaccaatgcataaggcattatgaaggcagcacagtccccgaaaagaag

SEQ ID NO: 12 - DNA, 63 bp, Homo sapiens
gtttctgggtgggggcgaaatgccaattttaaatttactgaccatctgaagtatgtcatgctg

SEQ ID NO: 13 - DNA, 387 bp, Homo sapiens
gtagactcaggcaatgatgtcacggatatcgcagatgacggctgcccgaagccccccgagattgcacatggctatgtggagcactcggttcgctaccagtgtaagaactactacaaactgcgcacagaaggagatggagtatacaccttaaatgataagaagcagtggataaataaggctgttggagataaacttcctgaatgtgaagcagatgacggctgcccgaagccccccgagattgcacatggctatgtggagcactcggttcgctaccagtgtaagaactactacaaactgcgcacagaaggagatggagtgtacaccttaaacaatgagaagcagtggataaataaggctgttggagataaacttcctgaatgtgaagca

SEQ ID NO: 14 - DNA, 210 bp, Homo sapiens
gtagactcaggcaatgatgtcacggatatcgcagatgacggctgcccgaagccccccgagattgcacatggctatgtggagcactcggttcgctaccagtgtaagaactactacaaactgcgcacagaaggagatggagtatacaccttaaatgataagaagcagtggataaataaggctgttggagataaacttcctgaatgtgaagca

SEQ ID NO: 15 - protein, 245 aa, Homo sapiens
ILGGHLDAKGSFPWQARMVSHHNLTTGATLINEQWLLTTAKMLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEENIWYATGILSFDKSCAVAEYGVYVKVTSIQDWVQKTIAEN

SEQ ID NO: 16 - protein, 82 aa, Homo sapiens
NYSQVDIGLIKLKQKVSVNERVMPICLPSKDYAEVGRVGYVSGWGRNANFKFTDHLKYVMLPVADQDQCIRHYEGSTVPEKK

SEQ ID NO: 17 - protein, 42 aa, Homo sapiens
VSGWGRNANFKFTDHLKYVMLPVADQDQCIRHYEGSTVPEKK

SEQ ID NO: 18 - protein, 21 aa, Homo sapiens
VSGWGRNANFKFTDHLKYVML

SEQ ID NO: 19 - protein, 129 aa, Homo sapiens
VDSGNDVTDIADDGCPKPPRIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEA

SEQ ID NO: 20 - protein, 70 aa, Homo sapiens
VDSGNDVTDIADDGCPKPPRIAHGYVEHSVRYQCKNYYKLRTEGDGVYTLNDKKQWINKAVGDKLPECEA